(19) United States
(12) Patent Application Publication
(10) Pub. No.: US 2009/0238852 A1
(43) Pub. Date: Sep. 24, 2009

(54) METHODS FOR CONTROLLING INTRACELLULAR CALCIUM LEVELS ASSOCIATED WITH AN ISCHEMIC EVENT

(75) Inventors: Thomas P. Kennedy, Charlotte, NC (US); William H. Barry, Salt Lake City, UT (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of Utah Research Foundation

(21) Appl. No.: 12/408,377

(22) Filed: Mar. 20, 2009

Related U.S. Application Data
(60) Provisional application No. 61/038,446, filed on Mar. 21, 2008.

(51) Int. Cl. A61F 2/04; A61K 31/727

(52) U.S. Cl. 424/423; 514/56

(57) ABSTRACT

Described herein are methods for controlling the intracellular calcium concentration in a subject prior to experiencing an ischemic event, while experiencing an ischemic event, or while suffering from ischemia. The methods comprise administering an effective amount of O-desulfated heparin to the subject. The methods described herein are also useful in treating the symptoms associated with ischemic events or ischemia.

### CROSS-REFERENCE TO RELATED APPLICATIONS

The present application claims the benefit of U.S. Provisional Patent Application No. 61/038,446, filed Mar. 21, 2008, the content of which is incorporated herein by reference in its entirety.

### BACKGROUND

The sodium-calcium (Na⁺/Ca⁺⁺) exchanger (NCE) provides homeostasis for intracellular levels of sodium (Na⁺) and calcium (Ca⁺⁺) (Blaustein M P, Lederer W J. Physiol Rev 79:763-854, 1999). Three isoforms of this exchange mechanism exist. Cardiac myocytes express primarily NCE1, while NCE2 and NCE3 are found primarily in brain and skeletal muscle (Nicoll D A, et al. Science 250:562-565, 1990; Li Z, et al. J Biol Chem 269:17434-17439, 1994; Nicoll D A, et al. J Biol Chem 271:24914-24921, 1996). During systole myocyte contraction is triggered by a sudden rise in intracellular Ca⁺⁺ concentration, but during diastole, intracellular myocyte Ca⁺⁺ concentrations must fall to enable cardiac relaxation. In cardiac muscle cells, or myocytes, NCE1 is found in the sarcolemmal membrane, where it provides the major route for Ca⁺⁺ extrusion from the cytosol. NCE1 accounts for 20-25% of the reduction of intracellular Ca⁺⁺ concentration during diastole. The remaining reduction of intracellular Ca⁺⁺ during diastole is provided by sequestration of Ca⁺⁺ by the sarcoplasmic Ca⁺⁺ ATPase (Blaustein M P, Lederer W J. ibid.). During removal of Ca⁺⁺ from the cytosol, NCE operates in the “forward” mode, exchanging three Na⁺ ions for one Ca⁺⁺ ion (3:1 stoichiometry). However, the Na⁺/Ca⁺⁺ is bidirectional and can operate in “reverse” mode with a change in ionic conditions. When this occurs, Na⁺ is extruded from the intracellular compartment in exchange for extracellular Ca⁺⁺, providing a pathologic mechanism by which intracellular Ca⁺⁺ concentration can rise precipitously, with disastrous consequences for the intracellular environment.

The most frequent pathologic situation where the NCE operates in reverse mode is in conditions of ischemia or ischemia followed by reperfusion. In these situations, the sudden inflow of Ca⁺⁺ from reverse mode operation of the NCE provokes sudden and disastrous events for the intracellular environment. In the case of myocardium, when reoxygenation begins to restore available energy in the form of adenosine triphosphate (ATP), high cytosolic calcium concentration ([Ca⁺⁺]) leads to uncontrolled activation of the contraction machinery (Piper H M, et al. Cardiovasc Res 61:365-371, 2004). This situation is most dramatically seen in cardiac surgery, where the heart is chemically stopped with cardioplegia solution for a period of minutes to hours while the surgeon replaces a heart valve or bypasses a coronary vessel. When the heart is restarted and coronary vessels are reperfused, reperfusion occasionally provokes the “stone heart” phenomenon, named because if the development of a stiff and pale heart resulting from massive muscle contracture that has occurred because of reverse mode NCE-mediated inflow of Ca⁺⁺, which triggers massive myocyte contraction. Under microscopy the stone heart demonstrates hypercontracted myofibrils and ruptured cellular membranes. A similar event also occurs in hearts during myocardial infarct from occlusion of a coronary artery, followed minutes to hours later by restoration of blood flow through the vessel either by dissolution of clot within the vessel by enzymatic digestion or by mechanical dissolution with the aid of an angioplasty catheter. During the earliest minutes of reperfusion, the region of ischemic and now reperfused myocardium undergoes a similar process of immediate hypercontracture as the initial and primary cause of cardiomyocyte necrosis. At the histologic level this is termed “contraction band necrosis” and is characterized by super-contracted sarcomeres and sarcolemmal disruption. The extent of contraction band necrosis correlates well with the degree of macroscopic myocardial shrinkage during the first minutes of myocardial reperfusion, and with the magnitude of enzyme release during the initial minutes of reflow. Hypercontracture leads to a rise in end-diastolic pressure and ventricular wall stiffness. That hyper-contracture itself is the destructive process has been demonstrated years ago in experiments showing that temporary contracture blockade of reperfused myocardium, applied for the first few minutes of reperfusion, can reduce the extent of developing injury and infarct size (Garcia-Dorado D, et al. Circulation 85:1160-1174, 1992; Siegmuind B, et al. Am J Physiol 260:H426-635, 1991).

When cells become ischemic, the cessation of blood flow and oxygen delivery impairs function of numerous ATP-dependent mechanisms that maintain normal Na⁺ and K⁺ concentrations by keeping Na⁺ out of the intracellular compartment. One target of ischemia is the Na⁺/K⁺ ATPase, which uses ATP to transport Na⁺ out of the cell in exchange for K⁺. This results in progressive accumulation of Na⁺ as the period of ischemia persists. The more important source of Na⁺ may be voltage-gated sodium channels (VGSCs). VGSC activation is triggered normally by membrane depolarization and results in the rapid influx of Na⁺ leading to further depolarization, Ca⁺⁺ entry and the initiation of excitation-contraction coupling. Normally, once activated, VGSCs rapidly inactivate, insuring that the influx of Na⁺ is transient. VGSC inactivation is slow or incomplete under some conditions, producing a sustained and persistent influx of Na⁺ referred to as late inward Na⁺ current I_{Na} (Noble D and Noble P J. Heart 92(Suppl4):iv1-5, 2006). Increased late Na⁺ current or I_{Na} is associated with inherited mutations in the VGSC causing long QT syndromes (Clancy C E, et al. J Clin Invest 110: 1251-1262, 2002) and can be induced by phosphorylation of VGSCs by stress-activated kinases (Light P E, et al., Circulation 107:1962-1965, 2003). Most importantly, VGSCs contribute to hypoxia-induced Na⁺ loading because I_{Na} is greatly augmented under conditions of ischemia (Ju Y K et al. J Physiol 497:337-347, 1996) or when cardiac myocytes are exposed to reactive oxygen species (Ward C A and Giles W R. J Physiol 500:631-642, 1997). These are the conditions present during myocardial ischemia and immediately after restoration of blood flow, when blood and oxygen delivery is interrupted for a variable period followed by restoration of flow through the coronary circulation. During myocardial ischemia from disruption of coronary flow, there is a steady rise in production of reactive oxygen species in ischemia cardiac myocytes. Thus, reactive oxygen species generation can account for much of the accumulation of Na⁺ during ischemia by augmentation of I_{Na}. With sudden return of coronary blood flow, production of reactive oxygen species rises dramatically in a burst of production that peaks within the next 5-6 minutes (Becker L B. Cardiovasc Res 61:461-470, 2004). Thus, reactive oxygen species augmentation of I_{Na} can continue to occur even after restoration of blood flow. Blocking Na⁺ accumulation with the Na⁺ channel inhibitor tetrodotoxin (TTX) prevents Na⁺ accumulation when cardiac myocytes are exposed to reactive oxygen species (Song Y, et al. J Pharmacol Exp Ther 318:214-222, 2006). Blocking Na⁺ accumulation with the Na⁺ channel inhibitor ranolazine prevents Na⁺ accumulation when hearts are exposed to ischemia (Fraser H, et al., J Mol Cell Cardiol 41:1031-1038, 2006).

The consequences of increased intracellular Na⁺ make themselves felt when ischemia is relieved by restoration of blood flow to the heart. When blood flow is restored, the high intracellular level of Na⁺ provokes reverse mode operation of the sarcolemmal NCE, resulting in the export of Na⁺, with a sudden spike in intracellular Ca⁺⁺ when ischemia has ended. Furthermore, the burst of reactive oxygen species occurring right at reperfusion will tend to greatly augment I_{Na} and promote additional Na⁺ accumulation intracellularly, further driving reverse mode operation of the NCE. If mitochondria are able to begin regeneration of ATP during the early phase of reperfusion when oxygen and nutrient delivery is restored, the high intracellular Ca⁺⁺ concentration can lead to uncontrolled myocyte contraction before restoration of the cellular energy state can lead to recovery from the loss of cytosolic cation balance. When calcium levels during reperfusion are analyzed in detail, they are found to rise and fall in spikes from cyclic uptake and release of Ca⁺⁺ by the sarcoplasmic reticulum, a complex network of anastamosing intracellular channels that surround the cardiac myofibrils. It is in the sarcoplasmic reticulum that Ca⁺⁺ is stored for release when myofibril contraction is to be initiated by an intracellular rise in Ca⁺⁺ concentration. It is also in the sarcolemmal membrane of the sarcoplasmic reticulum that the NCE resides. During reperfusion the oscillatory spikes in Ca⁺⁺ are promoted by ongoing Ca⁺⁺ influx across the sarcolemmal membrane through reverse mode NCE operation. These series of events are schematically depicted in FIG. 1. These events are not confined a single cardiac myocyte but envelope wide regions of cells that coordinate with one another through a system of gap-junction mediated communication, allowing the spread of cell injury during reperfusion (Garcia-Dorado D, et al. Circulation 96:3579-3586, 1997). The passage of Na⁺ through gap junctions from hypercontracting cells to adjacent relatively normal cells and the subsequent change in cytosolic Ca⁺⁺ levels through reverse mode operation of the NCE produces a propagation of hypercontracture in a wave spreading across the heart, thereby enlarging the area of injury. The importance of the NCE in this process of immediate injury is demonstrated by the protection of ischemic myocardium from ischemic contracture, functional decline in performance and tissue necrosis consequent to restoration of blood flow that is afforded by treatment of animal models with inhibitors of the NCE (Hagihara H, et al. Am J Physiol Heart Circ Physiol 288:H1699-H1707, 2005) or by genetic knockout of the NCE (Imahhashi K, et al. Circ Res 97:916-921, 2005). The essential role played by reverse mode operation of the NCE in Ca⁺⁺ accumulation in myocytes is further affirmed by the fact that NCE overexpression greatly enhances intracellular Ca⁺⁺ accumulation and myocyte injury in response to reactive oxygen species (Wagner S, et al. Cardiovasc Res 60:404-412, 2003), which, as already discussed, are produced in burst fashion in ischemic myocardium immediately after relief of ischemia. While not as well investigated, a similar mode of NCE-mediated, Ca⁺⁺-dependent immediate cellular injury occurs following ischemia in the central nervous system (Matsuda T, et al. J Pharmacol Exp Ther 298:249-256, 2001).

Oscillatory Ca⁺⁺ spikes mediating this process can be prevented by agents such as general anesthetics, which interfere with the sacroplasmic reticulum Ca⁺⁺ (Siegmuind B, et al. Circulation 96:4372-4379, 1997; Wickley P J, et al. Anesthesiology 106:302-311, 2007) or by inducing cellular acidosis (Ladilov Y V, et al. Am J Physiol 268:H1531-H1539, 1995; Schafer C, et al. Am J Physiol Heart Circ Physiol 278:H1457-H1463, 2000), which inhibits reverse mode NCE operation. Another method of preventing this process is with chemical inhibitors of reverse mode operation of the NCE. Presently, two such agents exist, the isothiourea derivatives (2-[2-[4-(4-nitrobenzyloxy)phenyl]ethyl]isothiourea metanesulfonate (KB-R7943) and 2-[4-[(2,5-difluorophenyl)methoxy]-phenoxy]-5-ethoxyaniline (SE0400). KB-R7943 has an NCE selectivity of 20 to 40 fold for NCE versus other ion channels and is 50-fold more selective for reserve mode versus forward mode (Iwamoto T, et al. J Biol Chem 271:22391-22397, 1996). SEA0400 has a higher NCE potency and greater selectivity than KB-R7943 against L-type Ca⁺⁺ channels, but lacks selectivity for the reverse versus forward mode of the NCE (Matsuda T, ibid.). Neither agent has been studied in human clinical trials, so that the toxicity of these isothiourea analogs is presently unknown in the setting of health or disease. Intracellular Ca⁺⁺ accumulation and myocyte necrosis can also be prevented during myocardial ischemia and reperfusion if hearts are pretreated prior to ischemia with the Na⁺ channel inhibitor ranolazine to prevent Na⁺ accumulation in myocytes during ischemia, thereby preventing the NCE from operating in reverse mode after ischemia is relieved (Hale S L, et al. J Pharmacol Exp Ther 3128:418-4223, 2006).

Recently, a novel and simple clinically applicable procedure has been discovered that reduces injury during the early reperfusion process. This procedure, termed “ischemic post-conditioning” is achieved by repetitive occlusion and reperfusion of the coronary artery in the early minutes after revascularization of acute myocardial infarction (Zhao Z Q, et al. Am J Physiol Heart Circ Physiol 285:H579-H588, 2003). Post-conditioning has been recently demonstrated in thirty patients submitted to coronary angioplasty for ongoing acute myocardial infarction. At the beginning of reperfusion by direct stenting, post-conditioning was performed within 1 minute of reflow by four episodes of one-minute inflation and one-minute deflation of the angioplasty balloon to produce four brief periods of ischemia and reflow. Compared to control subjects, this simple procedure produced a 36% reduction in infarct size as measured by the magnitude of cardiac enzyme release (Staat P, et al. Circulation 112:2143-2148, 2005). Ischemic post-conditioning has been shown to decrease myocyte injury by reducing intracellular Ca⁺⁺ overload during the early minutes of flow restoration (Sun H-Y, et al. Am J Physiol Heart Circ Physiol 288:H1900-H1908, 2005). The benefit of post-conditioning is dependent upon activation of the cardio-protective enzyme protein kinase Cε (PKCε), demonstrated in animal models by the fact that the infarct-sparing effect of postconditioning is abolished by PKCε inhibition (Zatta A J, et al. Cardiovasc Res 70:315-324, 2006). Because PKC enzymes can enhance both forward and reverse modes of NCE operation (Iwamoto T, et al. J Biol Chem 271:13609-13615, 1996), it is possible that ischemic post-conditioning functions to decrease myocardium Ca⁺⁺ overload by stimulation of PKCε-mediated phosphorylation of NCE in a differential fashion to decrease reverse and enhance forward NCE modes, providing an overall decrease in Ca⁺⁺ concentrations immediately after reperfusion. The initial reverse mode operation of the NCE during reperfusion may even be instrumental in activation of PKCε. In one recent study, inhibition of reverse mode operation of NCE by KB-R7943 or SEA0400 attenuated the pre-treatment protective effect of the general anesthetic sevoflurane against reperfusion related impairment in muscle contractility of isolated rat heart strips (Bouman R A, et al. Circulation 114-[suppl I]:I-226-I-232, 2006). Given its simplicity and potential for ready application with current angioplasty catheters, ischemic post-conditioning is likely to make its way into the practice of clinical interventional cardiology as a standard procedure following emergency stenting of coronary arteries for myocardial ischemia. It is therefore possible that the addition of other cardio-protective measures, such as blockade of reverse mode operation of the NCE, might provide additive benefit to ischemic post-conditioning alone, thereby reducing severity of myocardial injury more profoundly than could either strategy alone. In a preliminary report, the administration of the sodium/hydrogen exchanger inhibitor cariporide at the onset of reperfusion of ischemic rat hearts, in sequence with post-conditioning, reduced myocardial injury more substantially than did either procedure alone (Kin H, et al. Circulation 112:11-309, 2005). When combined with ischemic post-conditioning, a reverse mode NCE inhibitor might have to be administered at the end of the post-conditioning protocol in order to obtain maximal benefit from post-conditioning, if post-conditioning induced PKCε activation is indeed dependent upon reverse mode NCE operation during the post-conditioning protocol.

The sulfated polysaccharide heparin has been used in isolated cell patch clamp studies as an inhibitor of the intracellular calcium regulator molecule inositol triphosphate (IP3). Heparin binds to IP3 receptors, which act as intracellular Ca⁺⁺ on the endoplasmic reticulum membrane, and is an effective competitive antagonist with IP3 for these receptors (Ghosh T K, et al. J Biol Chem 263:11075-11079, 1988). Heparin is also a modulator of the ryanodine receptor, another type of intracellular Ca⁺⁺ (Bezprozvanny I B, et al. Mol Biol Cell 4:347-352, 1993). Finally, heparin can bind to and inhibit L-type Ca⁺⁺ channels (Lacinova, L, et al. J Physiol 465:181-201, 1993). All three of these effects are exhibited intracellularly, and require the microinjection of heparin into the isolated cell. Except for reticuloendothelial cells and endothelial cells which have active heparin uptake mechanisms, heparin is generally considered to be cell impermeate. Recently, heparin has been described to suppress Ca⁺⁺ in non-excitable HeLa cells when added to the external culture medium (Nemeth K, Kurucz I Biochem Pharmacol 69:929-940, 2005). However, the concentrations required for an effect were between 1.5 and 6.0 mg/ml. These concentrations would be unrealistic to achieve safely in patients. When heparin is used as an anticoagulant, therapeutic blood anticoagulation is achieved at heparin concentrations of less than about 1 U/ml. On a weight/volume basis (assuming 150 U/mg USP and anti-Xa anticoagulant activity for most commercial unfractionated heparin), therapeutic anticoagulation would then be achieved at a concentration of approximately 6 to 7 μg heparin per mL of blood. Increasing this concentration to even 1.5 mg (or 1,500 μg) per mL of blood would expose a patient to unconscionable levels of anticoagulation and risk of clinical bleeding. In a separate study, heparin and heparan sulfate derived two-sugar disaccharides added to the external culture medium have been recently reported to bind to the exchange inhibitor peptide of the NCE and reduce intracellular Ca⁺⁺ of smooth muscle cells in culture (Shinjo S K, et al. J Biol Chem 277:48227-48233, 2002). However, the effective dose for 50% reduction of intracellular Ca⁺⁺ (ED₅₀) was 88 μmol/L for the most potent disaccharide structure. When commercial heparin of approximately 12 kDa in size was studied, its ED₅₀ was found to be >5,000 μmol/L, which amounts to a concentration of >60 mg per mL. Such a high concentration of heparin would produce even greater degree of life-threatening anticoagulation.

Heparin has not been generally considered to block Na⁺ channels. In a recent electronic publication, the intracellular microinjection of heparin into oocytes was found to inhibit Na⁺ activity and intracellular Na⁺ accumulation under non-ischemic conditions (Bachhuber T, et al. J Biol Chem Published Feb. 28, 2008 as Manuscript M704532200. Available at http://wwwjbc.org/cgi/doi/10.1074/jbc.M704532200). Microinjection of heparin into the cells was required for this effect because oocytes do not readily take up and internalize heparin. Modification of Na⁺ channel activity or I_{Na} has not been reported to occur from heparin applied externally to the plasma membrane by addition to the medium.

A major problem in using heparin or heparin-derived agents to prevent injurious intracellular Ca⁺⁺ accumulation is that heparin and its derivatives cause heparin-induced thrombocytopenia (HIT), a disastrous fall in platelet count produced by the formation of a complex between heparin and platelet factor 4 (PF-4), a 70-amino acid platelet specific chemokine found in platelet granules. When heparin binds to PF-4, it produces a conformational change in PF-4, exposing an antigenic epitope to which some individuals have a circulating antibody (HIT antibody). The HIT antibody binds heparin-PF-4 complexes with high affinity. This antibody-heparin-PF-4 complex then binds to platelets by attachment of the antibody Fc domain to the platelet Fc receptor (FcγRIIa). This event in turn cross-links the Fc platelet receptors, inducing platelet activation and aggregation. A wave of platelet activation then ensues, producing consumption of platelets, a fall in platelet count to less than 50% of baseline (thrombocytopenia) and generalized coagulation, with potential development of life-threatening venous and arterial thrombosis, which can produce pulmonary embolism, myocardial infarction, stroke, or loss of limb perfusion. Any person receiving heparin or a heparin-like molecule is normally at risk for developing the type II heparin-induced thrombocytopenia that is associated with the risk of subsequent platelet-induced thrombosis. The overall risk for developing type II HIT is 0.5 to 3.0% of patients given heparin or a heparinoid (Chong, B H, et al., Expert Review of Cardiovascular Therapy 2:547-559, 2004).

### SUMMARY OF THE INVENTION

Described herein are methods for controlling the intracellular calcium ion concentration in a subject prior to experiencing or while experiencing an ischemic event or while suffering from ischemia. The methods may comprise administering an effective amount of an O-desulfated heparin (ODSH) or a derivative thereof to the subject.

In one embodiment, the inventive method comprises reducing the intracellular calcium ion concentration in a subject experiencing ischemia. In another embodiment, the method comprises maintaining the intracellular calcium ion concentration in a subject experiencing ischemia. In a further embodiment, the method comprises preventing an increase in the intracellular calcium ion concentration in a subject that is experiencing or is at risk of experiencing an ischemic event. In still another embodiment, the method comprises limiting an increase in the intracellular calcium ion concentration in a subject that is experiencing or is at risk of experiencing an ischemic event. Such various embodiments of control over the intracellular calcium ion concentration can be achieved by administering an effective amount of ODSH or a derivative thereof to the subject. In specific embodiments, the ODSH may be any of the following: heparin that is desulfated at the 2-O position, the 3-O position, or both the 2-O and 3-O positions; heparin that is fully desulfated at the 2-O position and partially desulfated at the 3-O position; heparin that is partially desulfated at the 2-O position and fully desulfated at the 3-O position; heparin that is substantially sulfated at the 6-O position; oxidized O-desulfated heparin; acetylated O-desulfated heparin; decarboxylated O-desulfated heparin; reduced O-desulfated heparin; 6-O desulfated heparin; or any combination of the foregoing.

The methods of the invention are useful across a broad range of events that may give rise to ischemia. Non-limiting examples of ischemic events that may be treatable according to the present invention include the following: (1) a surgical interruption of blood flow; (2) a pathologic acute or subacute arterial occlusion from thrombosis of a blood vessel; (3) ligation of the blood vessel or vascular remodeling and proliferative overgrowth within the vessel wall; (4) exposure to low concentrations of oxygen in the blood stream; (5) a reduction in blood pressure; (6) cardiopulmonary arrest; and (7) a low concentration of red blood cells within the circulation of the subject.

In specific embodiments, the methods of the invention may comprise controlling calcium ion concentration in myocytes, neurons, renal cells, hepatocytes, and lung cells of the subject. Control in such embodiments can include the types of control described above. In other embodiments, the inventive methods further may comprise reducing cellular influx of sodium ions.

The methods described herein are also useful in treating the symptoms associated with ischemic events or episodes of ischemia. Various examples of such symptoms are provided herein. In certain embodiments, the symptom may be selected from the group consisting of (1) pain from vascular occlusion or disruption, (2) tissue destruction from necrosis or apoptosis, (3) an impairment in organ function, (4) an abnormal rhythm disturbance, and (5) a neurological impairment. Further, the impairment in organ function particularly may be reduced during the ischemic event.

In some embodiments, the method of the invention also may comprise administering one or more further bioactive agents for treating or preventing the effects of the ischemic event or the ongoing ischemia. Such further bioactive agents can be administered sequentially or concurrently with the ODSH. Non-limiting examples of such further bioactive agents include a glycoprotein IIb/IIIa inhibitor, aspirin, clopidogrel, a thrombolytic agent, a tissue plasminogen activator, a tissue reteplase, a tissue tenecteplase, a direct thrombin inhibitor, a Na⁺ channel inhibitor, a form of activated protein C, a fully anticoagulant unfractionated or low molecular weight heparin, and combinations thereof.

In another embodiment, the invention is directed to a method for reducing the loss of function of a body part in a subject. The method may comprise administering an effective amount of an O-desulfated heparin to the subject prior to experiencing an ischemic event or while experiencing ischemia. In specific embodiments, the body part is an organ selected from the group consisting of the heart, brain, lung, bowel, and kidneys. In other embodiments, the body part is a body extremity (e.g., arm, leg, hand, foot, fingers, or toes).

The advantages of the invention will be set forth in part in the description which follows, and in part will be obvious from the description, or may be learned by practice of the aspects described below. The advantages described below will be realized and attained by means of the elements and combinations provided herein. It is to be understood that both the foregoing general description and the following detailed description are exemplary and explanatory only and are not restrictive.

### BRIEF DESCRIPTION OF THE DRAWINGS

FIG. 1 is a schematic depicting the pathogenesis of Ca⁺⁺ overload contracture of cardiac myocytes during the early minutes when ischemia is relieved.

FIG. 2 is a chemical formula of the pentasaccharide binding sequence of naturally occurring heparin, and the comparable sequence of 2-O, 3-O desulfated heparin (ODS heparin or ODSH).

FIG. 3 shows the effect of 2-O, 3-O desulfated heparin (ODSH) on intracellular calcium concentration [Ca⁺⁺]ᵢ in rabbit ventricular myocytes exposed to normal conditions (Hepes) or conditions of paced metabolic ischemia by culture under glucose-free conditions in a solution containing cyanide to impair mitochondrial and glycolytic generation of ATP.

FIG. 4 shows the effect of 2-O, 3-O desulfated heparin (ODSH, 100 μg/mL) and KB-R7943 (KBR, 10 μmol/L) on intracellular calcium concentration [Ca⁺⁺]ᵢ in rabbit ventricular myocytes exposed to normal conditions (Hepes) or conditions of paced metabolic ischemia as outlined in FIG. 3. *P<0.001 vs PMI alone.

FIG. 5 shows the effect of 2-O, 3-O desulfated heparin (ODSH) on intracellular sodium concentration [Na⁺]ᵢ in rabbit ventricular myocytes exposed to normal conditions (Hepes) or conditions of paced metabolic ischemia as outlined in FIG. 3.

FIG. 6 shows the effect of 2-O, 3-O desulfated heparin (ODSH) on intracellular calcium concentration [Ca⁺⁺]ᵢ in rabbit ventricular myocytes exposed to pacing in Hepes buffer (PH) with sea anemone toxin II (ATX) added to open cardiac myocyte membrane sodium channels. Pacing conditions were identical as in FIG. 3.

FIGS. 7A and 7B show the effects of ODSH on NCX current. FIG. 7A shows that ODSH (100 μg/ml) increased I_{NCX} over the voltage range of approximately −60 mV to +50 mV. FIG. 7B is a summary IV curve showing the stimulatory effect of ODSH on I_{NCX}. * P<0.05, n=5.

FIG. 8 is a graph showing the influence of ranolazine on [Ca²⁺]ᵢ during PMI, and on the effects of ODSH. Ranolazine (Ran, 10 μM) and ODSH 100 μm/ml caused a similar reduction in [Ca²⁺]ᵢ, and in the presence of ranolazine there was no further reduction in [Ca²⁺]ᵢ induced by exposure to ODSH. *P<0.05 vs PMI, n=7.

FIG. 9 is a graph showing the effect of ODSH on the rise in [Na⁺]ᵢ induced by exposure to sea anemone toxin II (ATX). Compared to control conditions (HEPES+pacing, HP) exposure to ATX 10 nM caused a highly significant increase in [Na⁺]ᵢ and this was reduced by exposure to 100 μm/ml ODSH. ODSH also caused a small but significant decrease in [Na⁺]ᵢ during control conditions (no ATX, HP alone). *P<0.05, **P<0.01 vs HP; ***P<0.01 vsHP+ATX, n=6.

FIG. 10 shows a graph of area at risk (AAR, left panel) and infarct size expressed as the area of necrosis relative to the AAR (NEC/AAR) as the consequence of administration of 2-O desulfated heparin to pigs in which the myocardium was made ischemic for 75 minutes (P<0.05 compared to control for ODS 15 and ODS 45), where the percentage values are mean±SE.

FIG. 11 is a graph showing myeloperoxidase activity (MPO) in ischemic-reperfused myocardium, expressed as Δabsorbance at 460 nm/minute/gram tissue (A460/min/g tissue). MPO was significantly reduced in the 45 mg/kg but not in 5 or 15 mg/kg ODSH groups. *P<0.05 vs other groups; ODSH 5 mg/kg (ODSH 5); ODSH 15 mg/kg (ODSH 15); ODSH 45 mg/kg (ODSH 45).

FIG. 12 shows activated clotting times (ACT) during the course of administration of 2-O desulfated heparin to pigs in which the myocardium was made ischemic for 75 minutes.

FIG. 13 shows cross-reactivity of the 2-O desulfated heparin lot HM0506394 of this invention to heparin antibody, as determined by the serotonin release assay.

FIG. 14 shows cross-reactivity of the 2-O desulfated heparin lot HM0506394 of this invention to heparin antibody, as determined by expression of platelet surface P-selectin (CD62) quantitated by flow cytometry.

FIG. 15 is a graph of mean plasma concentrations of 2-O desulfated heparin in normal human subjects receiving a bolus dose of this agent intravenously.

FIG. 16 is a graph of mean change from baseline in activated partial thromboplastin time (aPTT) in normal human subjects receiving an intravenous bolus dose of 2-O desulfated heparin.

FIG. 17 is a graph of mean change from baseline in activated clotting time (ACT) in normal human subjects receiving an intravenous bolus dose of 2-O desulfated heparin.

FIG. 18 is a graph of mean plasma concentrations of 2-O desulfated heparin in normal human subjects receiving a bolus followed by 12 hour infusion of drug.

FIG. 19 is a graph of mean change from baseline in activated partial thromboplastin time (aPTT) in normal human subjects receiving an intravenous bolus dose and 12 hour infusion of 2-O desulfated heparin.

FIG. 20 is a graph of mean change from baseline in activated clotting time (ACT) in normal human subjects receiving an intravenous bolus dose and 12 hour infusion of 2-O desulfated heparin.

FIG. 21 is a graph of mean plasma levels of 2-O desulfated heparin (ODSH) in subjects receiving an intravenous bolus of 8 mg/kg O-desulfated heparin followed by an infusion of 0.6 mg/kg/hr for 72 hours, titrated to maintain aPTT at the upper limit of normal (ULN) in the range of 40-45 seconds.

FIG. 22 is a graph of mean activated partial thrombopastin time (aPTT) in normal human subjects receiving an intravenous bolus of 8 mg/kg 2-O desulfated heparin followed by an infusion of 0.6 mg/kg/hr for 72 hours, titrated to maintain aPTT at the upper limit of normal (ULN) in the range of 40-45 seconds.

FIG. 23 is a graph showing the relationship between plasma levels of 2-O desulfated heparin (ODSH) and change in activated partial thromboplastin time (aPTT) from baseline in normal human subjects receiving an intravenous bolus of 8 mg/kg O-desulfated heparin followed by an infusion of 0.6 mg/kg/hr for 72 hours, titrated to maintain aPTT in the upper limit of normal (ULN) in the range of 40-45 seconds.

FIG. 24 is a series of graphs showing the effects of ODSH on Na⁺ channel ionic currents. FIG. 24A shows peak Na⁺ current-voltage relationships from a holding potential of −150 mV. Open circles are values in presence of 1 mg/ml ODSH heparinic acid. All Na⁺ currents were normalized to the maximal inward I_{Na} in control. The lines represent the fits to the Boltzmann equation for peak IV relationships. FIG. 24B shows peak I-V relationships from a holding potential of −110 mV for control (closed circles) and in 1 mg/ml ODSH heparinic acid (open circles). All Na currents were normalized to the maximal inward I_{Na} in control. The lines represent the fits to the Boltzmann equation for peak IV relationships. FIG. 24C shows steady-state voltage-dependent Na⁺ channel availability (SSI) curves in control (closed circle) and in 1 mg/ml ODSH heparinic acid (open circles). All Na⁺ currents in each cell were normalized to its Iₘₐₓ from the fit of a Boltzmann relationship to SSI curve in control. The lines represent the fits to the Boltzmann equation. FIG. 24D shows late I_{Na} determined by STX substraction of leak currents from a holding potential of −110 mV to step potentials from −100 to 20 mV for 100 msec. The closed circles represent the means (±SEM) I_{Na} in control while the open circles represent the means (±SEM) in ODSH heparinic acid for four cells.

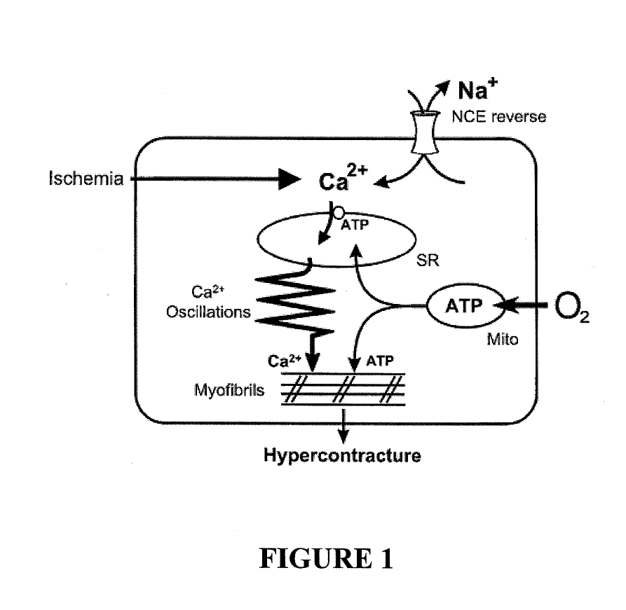

### DETAILED DESCRIPTION OF THE INVENTION

Before the present compounds, compositions, articles, devices, and/or methods are disclosed and described, it is to be understood that the aspects described below are not limited to specific compounds, synthetic methods, or uses and as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally bioactive agent” means that the bioactive agent may or may not be present.

### I. Active Agents

The present invention provides pharmaceutical compositions useful in methods of preventing or reducing dangerous Ca⁺⁺ buildup within the ischemic cell by blocking Na⁺ channels and preventing elevated intracellular Na⁺ accumulation that drives reverse mode operation of the NCE The pharmaceutical compositions of the invention generally include O-desulfated heparin (ODSH) as an active agent. In certain embodiments, the pharmaceutical compositions can include one or more further active agents.

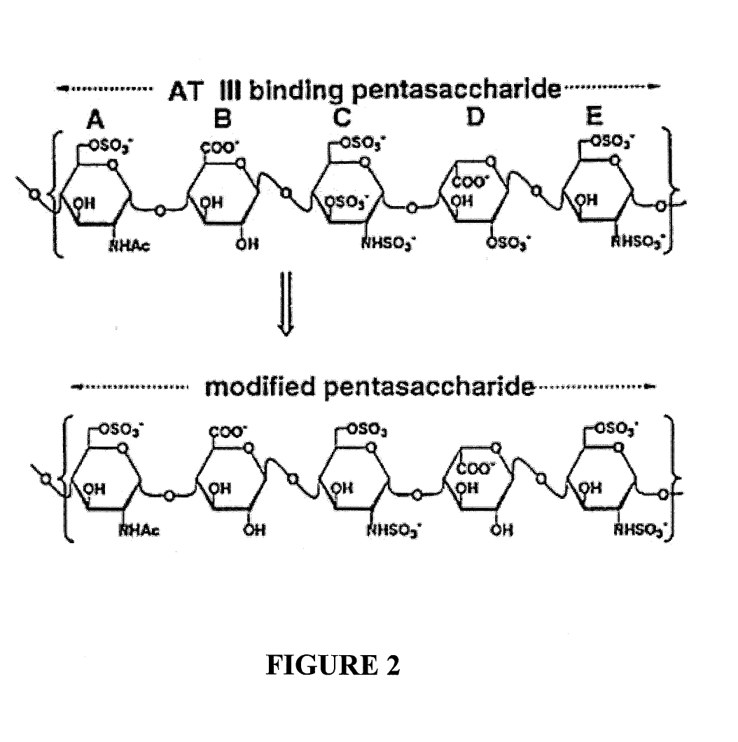

The chemical formula of naturally occurring heparin is shown in FIG. 2. The term “O-desulfated heparin” refers to heparin that has been modified to remove at least a portion of the O-sulfate groups therefrom. Preferably, the term refers to heparin that is O-desulfated sufficiently to have resulted in any reduction of the anticoagulant activity of the heparin. In specific embodiments, the O-desulfated heparin is at least partially, and preferably substantially, desulfated at least at the 2-O position, at least at the 3-O position, or at both the 2-O position and the 3-O position.

In preferred embodiments, the O-desulfated heparin is at least about 10%, at least about 25%, at least about 50%, at least about 75%, at least about 80%, at least about 90%, at least about 95%, at least about 97%, or at least about 98% desulfated, independently, at each of the 2-O position and the 3-O position. In specific embodiments, the O-desulfated heparin is 100% desulfated at one or both of the 2-O and the 3-O position. The extent of O-desulfation need not be the same at each O-position. For example, the heparin may be predominately (or completely) desulfated at the 2-O position and have a lesser degree of desulfation at the 3-O position. In one embodiment, the O-desulfated heparin includes 2-O, 3-O desulfated heparin, wherein the heparin is at least about 90% desulfated at both the 2-O and 3-O positions. The O-desulfated heparins synthesized and disclosed in U.S. Pat. Nos. 6,489,311; 6,077,683; 5,990,097; 5,668,118; and 5,707,974 can be used herein.

The extent of O-desulfation or N-desulfation can be determined by known methods, such as disaccharide analysis. Although 6-O desulfation cannot be determined by currently available techniques, in a preferred embodiment, the 6-O position is substantially sulfated. For example, the 6-O position is at least about 80%, at least about 85%, at least about 90%, at least about 95%, at least about 99%, or 100% sulfated. Of course, the invention still encompasses heparin wherein some, particularly a minor amount, of the 6-O sulfates were lost (desulfated) during the preparation of the compounds used in the invention. N-sulfates are generally stable under alkaline hydrolytic conditions. Thus, in certain embodiments, the heparin used according to the invention can have most of its N-sulfate groups remaining intact. Of course, the invention does encompass heparin having some of the N-sulfates removed.

One method of preparing O-desulfated heparin is provided in U.S. Pat. No. 5,990,097, which is described above. In the method disclosed therein, a 5% aqueous solution of porcine intestinal mucosal sodium heparin is made by adding 500 gm heparin to 10 L deionized water. Sodium borohydride is added to a 1% final concentration and the mixture is incubated. Sodium hydroxide is then added to a 0.4 M final concentration (pH at least 13) and the mixture is frozen and lyophilized to dryness. Excess sodium borohydride and sodium hydroxide can be removed by ultrafiltration. The final product is pH adjusted, cold ethanol precipitated, and dried. The O-desulfated heparin produced by this procedure is a fine crystalline slightly off-white powder with less than 10 USP units/mg anti-coagulant activity and less than 10 U/mg anti-Xa anti-coagulant activity.

The synthesis of O-desulfated heparin as described above can also include various modifications. For example, the starting heparin can be placed in, for example, water, or other solvent, as long as the solution is not highly alkaline. A typical concentration of heparin solution can be from 1 to 10 percent by weight heparin. The heparin used in the reaction can be obtained from numerous sources, known in the art, such as porcine intestine or beef lung. The heparin can also be modified heparin, such as the analogs and derivatives described herein.

The heparin can be reduced by incubating it with a reducing agent, such as sodium borohydride, catalytic hydrogen, or lithium aluminum hydride. A preferred reduction of heparin is performed by incubating the heparin with sodium borohydride. Generally, about 10 grams of NaBH₄ can be used per liter of solution, but this amount can be varied as long as reduction of the heparin occurs. Additionally, other known reducing agents can be utilized but are not necessary for producing a treatment effective O-desulfated heparin. The incubation can be achieved over a wide range of temperatures, taking care that the temperature is not so high that the heparin caramelizes. Exemplary temperature ranges are about 15-30° C. or about 20-25° C. The length of the incubation can also vary over a wide range, as long as it is sufficient for reduction to occur. For example, several hours to overnight (i.e., about 4 to 12 hours) can be sufficient. However, the time can be extended to over several days, for example, exceeding about 60 hours.

Additionally, the method of synthesis can be adapted by raising the pH of the reduced solution to 13 or greater by adding a base to the reduced heparin solution, wherein the base is capable of raising the pH to 13 or greater. The pH can be raised by adding any of a number of agents including hydroxides, such as sodium, potassium or barium hydroxide. A preferred agent is sodium hydroxide (NaOH). Even once a pH of 13 or greater has been achieved, it can be beneficial to further increase the concentration of the base. For example, it is preferable to add NaOH to a concentration of about 0.25 M to about 0.5 M NaOH. This alkaline solution may then be dried, lyophilized or vacuum distilled.

In specific embodiments, the alkaline solution can include heparin and base in defined ratios. For example, when NaOH is used as the base, the ratio of NaOH to heparin (NaOH:heparin, in grams) can be about 0.5:1, preferably about 0.6:0.95, more preferably about 0.7:0.9. Of course, greater concentrations of base can be added, as necessary, to ensure the pH of the solution is at least 13.

Heparin is a heterogeneous mixture of variably sulfated polysaccharide chains composed of repeating units of D-glucosamine and either L-iduronic acid or D-glucuronic acids. The average molecular weight of heparin typically ranges from about 6,000 Da to about 30,000 Da, although certain fractions of unaltered heparin can have a molecular weight as low as about 1,000 Da. According to certain embodiments of the invention, heparin can have a molecular weight in the range of about 1,000 Da to about 30,000 Da, about 3,000 Da to about 25,000 Da, about 8,000 Da to about 20,000 Da, or about 10,000 Da to about 18,000 Da. Unless otherwise noted, molecular weight is expressed herein as weight average molecular weight (M_{w}), which is defined by formula (I) below

$${M}_{W} = \frac{\sum {n}_{i} {M}_{i}^{2}}{\sum {n}_{i} {M}_{i}} , \qquad ( I )$$

wherein nᵢ is the number of polymer molecules (or the number of moles of those molecules) having molecular weight Mᵢ.

The O-desulfated heparin used according to the invention can also have a reduced molecular weight so long as it retains the useful activity as described herein. Low molecular weight heparins can be made enzymatically by utilizing heparinase enzymes to cleave heparin into smaller fragments, or by depolymerization using nitrous acid. Such reduced molecular weight O-desulfated heparin can typically have a molecular weight in the range of about 100 Da to about 8,000 Da. In specific embodiments, the heparin used in the invention has a molecular weight in the range of about 100 Da to about 30,000 Da, about 100 Da to about 20,000 Da, about 100 Da to about 10,000 Da, about 100 to about 8,000 Da, about 1,000 Da to about 8,000 Da, about 2,000 Da to about 8,000 Da, or about 2,500 Da to about 8,000 Da. Preferably, the average molecular weight of the heparin after O-desulfation is in the range of about 4,000 to about 12,500 Da.

The O-desulfated heparin used according to the present invention can be in any form useful for delivery to a patient provided the O-desulfated heparin maintains the activity useful in the methods of the invention, particularly the low anticoagulation activity of the O-desulfated heparin. Non-limiting examples of further forms the O-desulfated heparin may take on that are encompassed by the invention include esters, amides, salts, solvates, prodrugs, or metabolites. Such further forms may be prepared according to any methods that are known in the art, such as, for example, those methods described by J. March, Advanced Organic Chemistry: Reactions, Mechanisms and Structure, 4^{th} Ed. (New York: Wiley-Interscience, 1992), which is incorporated herein by reference.

As noted above, in certain embodiments, the compositions for use according to the methods of the invention can include one or more active agents in addition to O-desulfated heparin. Non-limiting examples of active agents that can be combined with O-desulfated heparin for treatment of ischemia and ischemic related reverse mode operation of the NCE to prevent intracellular Ca⁺⁺ overload include any drugs presently used in management of ischemia generally or for treatment of ischemia. For example, the O-desulfated heparin may be combined with one or more glycoprotein IIb/IIIa inhibitors such as tirofiban hydrochloride, eptifibatide or abciximab, with aspirin and/or clopidogrel, with thrombolytic agents such as streptokinase, tissue plasminogen activator, reteplase or tenecteplase, with direct thrombin inhibitors such as argatroban or lepirudin, with the Na⁺ channel inhibitor ranolazine, with forms of activated protein C such as drotecogin alfa, with fully anticoagulant unfractionated or low molecular weight heparins, as an adjunctive measure in treating cardiopulmonary arrest, with rescue angioplasty and/or stent placement in an occluded artery, with protocols for ischemic pre-conditioning or post-conditioning of an organ, with coronary artery bypass or valvular surgery, with cardiopulmonary bypass, with vascular procedures such as carotid endarterectomy, repair of an aortic aneurysm or femoral-popliteal bypass, with inhibitors of protein kinase C delta or activators of protein kinase C epsilon, with supplemental or hyperbaric oxygen therapy, or with pressor therapy for low blood pressure. Of course, such disclosure should not be viewed as limiting the scope of further active agents that may be combined with O-desulfated heparin. Rather, any further compounds generally recognized as useful for treating ischemia, blocking Na⁺ channels or I_{Na}, inhibiting reverse mode operation of the NCE or reducing intracellular Ca⁺⁺ overload may be used in addition to the compounds specifically noted herein.

### II. Methods of Treatment

The present invention generally provides methods of treatment of subjects experiencing an ischemic condition or event, at risk of experiencing an ischemic event, or suffering from ischemia. In particular, the invention relates to ischemic events that induce or tend to cause injurious increases in the intracellular Ca⁺⁺ concentration. Intracellular Ca⁺⁺ concentration may be monitored by relative fluorescence of a detector molecule that is sensitive to intracellular calcium. See for example, Y. V. Ladilov et al., Protection of Reoxygenated Cardiomyocytes Against Hypercontracture by Inhibition of Na⁺/H⁺ Exchange, Am. J. Physiol. 268:H1531-9 (1995), incorporated herein by reference. The values provided by such measurements are relative rather than absolute. Nevertheless, such method would be expected to provide a reliable method for evaluating intracellular Ca⁺⁺ concentration in an individual subject or in an entire class of subjects to determine a baseline concentration prior to experiencing an ischemic event, to determine changes in concentration during an ischemic event, to determine whether an increase in concentration has occurred as a result of an ischemic event, and to monitor ongoing concentration during an episode of ischemia.

In general, preventing calcium overload (i.e., increased intracellular concentrations) prior to or during an ischemic event will allow ischemic tissue to safely restore its energy state and proper ionic membrane gradients without undergoing destructive processes induced by elevated amounts of intracellular Ca⁺⁺. As used herein, ischemia is understood to mean an insufficient supply of blood to an organ or tissue of a subject, and is generally produced by the interruption of blood supply to that organ or tissue. As used herein, ischemic event is understood to mean any instance that results, or could result, in a deficient supply of blood to the tissues of the CNS, including the brain and/or spinal cord. Ischemic events encompassed by the present invention include, but are not limited to, stroke, such as stroke caused by emboli within cerebral vessels, arteriosclerotic vascular disease, the inflammatory processes, which frequently occur when thrombi form in the lumen of inflamed vessels, or hemorrhage; multiple infarct dementia; cardiac failure and cardiac arrest; shock, including septic shock and cardiogenic shock; blood dyscrasias; hypotension; hypertension; an angioma; hypothermia; perinatal asphyxia; high altitude ischemia; hypertensive cerebral vascular disease; rupture of an aneurysm; seizure; bleeding from a tumor; and traumatic injury to the central nervous system, including open and closed head injury, neck injury, and spinal cord trauma such as occurs with a blow to the head, neck, or spine, or with an abrasion, puncture, incision, contusion, compression, and the like in any part of the head, neck, or vertebral column. Ischemia can also be induced by exposure to low concentrations of oxygen in the blood stream, as might occur with high altitude or with lung dysfunction sufficiently severe so that proper oxygenation of the arterial blood fails to occur. Other possible ischemic events include traumatic injury due to constriction or compression of CNS tissue by, for example, subdural or intracranial hematoma, by a mass of abnormal tissue, such as a metastatic or primary tumor, by over accumulation of fluid, such as cerebrospinal fluid as a result of dysfunction of normal production, or by edema. A mammal subject to the above conditions may be considered to be at risk for experiencing an ischemic event.

A mammal particularly may be at risk of experiencing an ischemic event for medical or other reasons. For example, a mammal undergoing a cardiovascular surgical procedure, including, but not limited to, by-pass surgery, open-heart surgery, aneurysm surgery, surgery on a major vessel, pathologic acute or subacute arterial occlusion from thrombosis of a blood vessel, ligation of the blood vessel or vascular remodeling and proliferative overgrowth within the vessel wall which encroaches upon the vascular lumen, and cardiac catheterization whether for treatment or diagnostic purposes may be at risk during or following the procedure. A mammal with a medical condition may be at risk of experiencing an ischemic event. Such medical conditions include, but are not limited to, herpes meningitis; hypertensive encephalopathy; myocardial infarction; and edema within a CNS tissue, such as results with viral infection or traumatic injuries noted above. Ischemia likewise can result from overall lowering of blood pressure to such a point as the organism is not adequately perfused with blood, as in many forms of arterial shock from hemorrhage or infection, or from cardiopulmonary arrest. Furthermore, ischemia of tissues can result from abnormally low concentrations of red blood cells within the circulation, such as in anemia, and can occur when the subject is poisoned with inhibitors of mitochondrial function such as cyanide or carbon monoxide. Thus, the methods of the invention can mitigate or alleviate ischemia, such as ischemia arising from an ischemic event as described above. The methods of the invention may also be used to treat one or more symptoms arising from an ischemic event.

In one embodiment, the methods described herein are particularly useful for relieving symptoms of acute ischemia. In one embodiment, the invention is useful for relieving ischemic pain, particularly pain from vascular occlusion or disruption. In other embodiments, the method is useful for treating tissue destruction form necrosis or apoptosis resulting from ischemia as the consequence of tissue overload from Ca⁺⁺. The inventive method is further useful for preventing impairment in organ function from ischemia-induced Ca⁺⁺ overload. For example, it may prevent impairment in organs including the heart, brain, lung, bowel, and kidneys. In still further embodiments, the method of the invention is useful for preventing abnormal rhythm disturbances consequent to ischemia, when the treated organ is the heart.

The methods of treatment according to the invention generally include administering O-desulfated heparin to a patient prior to experiencing an ischemic event, while experiencing an ischemic event, or while suffering ischemia, placing him at risk from reverse mode operation of the NCE. Such ischemia can be determined by the presence of one or more of the symptoms of ischemia, including abnormal temperature of the organ, pain in the ischemic organ, shortness of breath from abnormal organ performance if the affected organ is the heart, mental or other neurologic abnormalities from discrete structural or global ischemia if the affected organ is the brain. These and many other symptoms can occur specific to the organ or organs involved with ischemic condition, as well as any further symptoms generally recognized as signaling ischemia of an organ or of the entire subject or patient.

The methods of the present invention particularly can control the intracellular calcium ion concentration in a subject prior to experiencing an ischemic event or while experiencing ischemia. “Controlling,” as used herein, can mean any of the following: reducing intracellular calcium ion concentrations in a subject experiencing ischemia, maintaining intracellular calcium ion concentrations in a subject experiencing ischemia, preventing an increase in calcium ion concentration in a subject experiencing an ischemic event or at risk of experiencing an ischemic event, or limiting the increase in calcium ion concentration in a subject experiencing an ischemic event or at risk of experiencing an ischemic event.

Reducing intracellular calcium ion concentrations in a subject suffering from ischemia may particularly refer to a reduction in intracellular calcium ion concentration when a subject experiencing ischemia is administered an O-desulfated heparin compared to the intracellular calcium ion concentration of the same subject experiencing ischemia but not administered the O-desulfated heparin. The amount of reduction can vary. For example, the amount of reduction can be up to about 5%, up to about 10%, up to about 20%, up to about 30%, up to about 40%, or up to about 50%. In some embodiments, the amount of reduction in intracellular calcium ion concentration can be from 1-50%, 5-40%, or 10-30%.

Maintaining intracellular calcium ion concentrations in a subject suffering from ischemia may particularly refer to maintaining the intracellular calcium ion concentration at the same or a similar concentration as prior to experiencing the ischemic event when a subject experiencing ischemia is administered an O-desulfated heparin, compared to the intracellular calcium ion concentration of the same subject experiencing ischemia but not administered the O-desulfated heparin. The amount of change in the concentration can vary. For example, the amount of reduction can be within 10%, 5%, 2%, or less than 1% from the initial intracellular calcium ion concentration. In some embodiments, the amount intracellular calcium ion concentration is maintained at from 0-10%, 0-5%, or 0-1%. Thus, the term “maintain” includes slight increases in intracellular calcium ion concentrations.

Preventing an increase in calcium ion concentration in a subject experiencing an ischemic event or at risk of experiencing an ischemic event may particularly refer to the ability of the O-desulfated heparin to maintain intracellular calcium ion concentrations within 10%, 5%, 2%, or less than 1% from the initial intracellular calcium ion concentration prior to the ischemic event. Thus, the term “prevent” includes slight increases in intracellular calcium ion concentrations. For example, the O-desulfated heparin can be administered prior to exposure to an ischemic stimulus such as, for example, a scheduled surgery or exposure to altitude or low environmental oxygen. The term “prevention” with respect to treating one or more symptoms produced by an ischemic event is defined herein as either substantially reducing the severity of the symptom or preventing the occurrence of the symptom completely.

Preferably a prevention method of this invention has a constant suppression of ischemic-related organ or tissue Ca⁺⁺ overload, which can be achieved by a repetitive, routine administration of the O-desulfated heparin. With repetitive, routine administration, an optimal dose can readily be ascertained by varying the dose until the optimal prevention is achieved. Additionally, upon exposure to organ or whole body ischemia, if eventually one or more symptoms of ischemia occur, an additional dose of O-desulfated heparin can be administered. Additionally, when an exposure to an ischemic event is known in advance, an additional dose of O-desulfated heparin can be administered to prevent a response.

Limiting the increase in calcium ion concentration in a subject experiencing an ischemic event or at risk of experiencing an ischemic event may particularly refer to reducing or lowering the increase in intracellular calcium ion concentration prior to an ischemic event or during an ischemic event. For example, upon administration of an effective amount of an O-desulfated heparin or a derivative thereof to the subject, the rate and amount of increase in intracellular calcium ion concentration is lower when compared to the same subject who was not administered the O-desulfated heparin or a derivative thereof.

In one embodiment, the method of the invention is used to control the intracellular calcium ion concentration in myocytes or neurons, in a subject prior to or while experiencing an ischemic event by administering an effective amount of O-desulfated heparin or a derivative thereof to the subject. Changes in the intracellular calcium ion concentrations in cardiac myocytes and neurons may result from myocardial infarction and stroke, two of the major diseases consequent to ischemia reperfusion injury. Accumulation of intracellular calcium is likely to lead to cell death.

In another embodiment, the method of the invention can be used to control the intracellular calcium ion concentration in specific cell types. Non-limiting examples of the types of cells with which the method can be used include renal cells, hepatocytes, and lung cells. An effective amount of ODSH may be administered to these cells in vitro or in vivo to provide the desired effect. In one embodiment, the ODSH can be administered to the cells as a component of an organ preservation solution (i.e., a solution used to flush organs in order to remove blood and stabilize the organs for the time required for organ allocation, transportation, transplantation or the like). In other embodiments, the ODSH could be administered directly to cells and/or directly to a functioning organ for the purpose of providing the desired effect on the specified cell types.

While not wishing to be bound by theory, it is believed that the use of desulfated heparin according to the invention is particularly useful since it blocks the influx of Na⁺ into the cell, thereby reducing secondary Ca⁺⁺ overload in ischemic tissue or organs possibly by preventing elevated intracellular Na⁺ concentrations that might stimulate reverse mode operation of the NCE.

In certain embodiments, the invention is directed to methods of reducing organ injury due to loss of function from ischemic-induced tissue destruction from necrosis or apoptosis. The present invention is particularly useful in that the methods of treatment described herein can significantly reduce organ injury from ischemic-related organ or tissue Ca⁺⁺ overload. Specifically, the invention may reduce the loss of organ function during an ischemic event. The organ may be, but is not limited to, the heart, brain, lung, bowel, and kidneys. This is highly beneficial not only from the standpoint of reduced loss of organ function to the patient, but also for improving patient quality of life from restoration of proper organ function once the ischemic process is relieved by other methods.

The Examples provided below illustrate the ability of the inventive methods for reducing ischemic-related organ or tissue Ca⁺⁺ overload. This is particularly so for patients treated with conventional therapies in association with the treatments of the invention. In particular, the method for reducing ischemic-related organ or tissue Ca⁺⁺ overload includes administering to the patient a pharmaceutical composition having an amount of O-desulfated heparin effective to reduce or treat the tissue Ca⁺⁺ overload. Such treatment with ODSH allows for recovery of the ischemic organ that is greater than that which would be experienced without treatment with the ODSH (including patients treated with the conventional therapies of ischemia).

In light of the above, it is clear that the methods of the invention, including treatment with ODSH, hasten the time to improvement of the ischemic organ, including when added to the conventional standard of care therapy for such patients. Accordingly, the invention can provide methods of reducing the loss of organ function. In specific embodiments, treatment with ODSH according to the present invention reduces the loss of function of an ischemic organ by at least about 10% compared to a patient suffering ischemia but not treated with ODSH. In further embodiments, the loss of organ function is reduced by at least about 15%, at least about 20%, at least about 25%, at least about 30%, at least about 40%, at least about 50%, or at least about 60%. In further embodiments, the reduced organ injury can be described in terms of the performance of the organ. In specific embodiments, treatment according to the invention reduces the depression in cardiac ejection fraction as measured by ultrasonic echocardiography or nuclear medicine functional scanning techniques. In still other embodiments, the invention is directed to methods for treating the abnormal cardiac rhythms resulting from ischemia and/or occurring when ischemia is relieved.

In further embodiments, the neurologic impairment from central nervous system ischemia is reduced so that the patient have better motor and sensory function than if not treated with the invention herein. In still additional embodiments, the subjects treated herein recovers from carbon monoxide or cyanide poisoning with reduced cognitive impairment compared to individuals treated conventionally for these poisoning. In still further embodiments, subjects treated herein and undergoing aortic aneurysm repair or similar vascular surgeries will experience less post-operative edema of extremities distal to vascular clamp occlusion during the operation, and will have fewer complicating organ dysfunctions such as acute lung injury. For example, the desulfated heparin can be administered at the time of primary PCI for patients with acute coronary artery occlusion and ST elevation myocardial infarction. In yet additional embodiments, subjects treated herein prior to and during cardiac surgery will have improved myocardial performance after surgery is completed and not be at risk for development of the “stone heart” phenomenon when cardiac bypass is discontinued. In additional embodiments, subjects treated herein immediately upon the experience of cardiopulmonary arrest will have a greater recovery with decreased brain and other organ impairment after normal cardiopulmonary function is restored.

### III. Biologically Active Variants

Biologically active variants of O-desulfated heparin are particularly also encompassed by the invention. Such variants should retain the biological activity of the original compound; however, the presence of additional activities would not necessarily limit the use thereof in the present invention. Such activity may be evaluated using standard testing methods and bioassays recognizable by the skilled artisan in the field as generally being useful for identifying such activity.

According to one embodiment of the invention, suitable biologically active variants include analogues and derivatives of the compounds described herein. Indeed, a single compound, such as those described herein, may give rise to an entire family of analogues or derivatives having similar activity and, therefore, usefulness according to the present invention. Likewise, a single compound, such as those described herein, may represent a single family member of a greater class of compounds useful according to the present invention. Accordingly, the present invention fully encompasses not only the compounds described herein, but analogues and derivatives of such compounds, particularly those identifiable by methods commonly known in the art and recognizable to the skilled artisan. An analog is defined as a substitution of an atom or functional group in the heparin molecule with a different atom or functional group that usually has similar properties. A derivative is defined as an O-desulfated heparin that has another molecule or atom attached to it.

In certain embodiments, an analog of O-desulfated heparin, as described herein, includes compounds having the same functions as O-desulfated heparin for use in the methods of the invention (including minimal anticoagulant activity), and specifically includes homologs that retain these functions. For example, various substituents on the heparin polymer can be removed or altered by any of many means known to those skilled in the art, such as acetylation, deacetylation, decarboxylation, oxidation, reduction, etc., so long as such alteration or removal does not substantially increase the low anticoagulation activity of the O-desulfated heparin. Any analog can be readily assessed for these activities by known methods given the teachings herein.

The O-desulfated heparin of the invention may particularly include O-desulfated heparin having modifications, such as reduced molecular weight or acetylation, deacetylation, oxidation, decarboxylation, or reduction as long as it retains its ability to function according to the methods of the invention. Such modifications can be made either prior to or after partial desulfation and methods for modification are standard in the art. As noted above, the O-desulfated heparin can particularly be modified to have a reduced molecular weight, and several low molecular weight modifications of heparin have been developed (see page 581, Table 27.1 Heparin, Lane & Lindall). In one aspect, a derivative of the O-desulfated heparin includes N-desulfated heparin, N-desulfated N-acetylated heparin, N-decarboxylated heparin, 6-O desulfated heparin, carboxy-reduced heparin, periodate oxidized heparin, periodate oxidized sodium borohydride reduced heparin, or a low molecular weight species of these derivatives.

Periodate oxidation (U.S. Pat. No. 5,250,519, which is incorporated herein by reference) is one example of a known oxidation method that produces an oxidized heparin having reduced anticoagulant activity. Other oxidation methods, also well known in the art, can be used. Additionally, for example, decarboxylation of heparin is also known to decrease anticoagulant activity, and such methods are standard in the art. Furthermore, some low molecular weight heparins are known in the art to have decreased anti-coagulant activity, including Vasoflux, a low molecular weight heparin produced by nitrous acid depolymerization, followed by periodate oxidation (Weitz J I, Young E, Johnston M, Stafford A R, Fredenburgh J C, Hirsh J. Circulation. 99:682-689, 1999). Thus, modified O-desulfated heparin (or heparin analogs or derivatives) contemplated for use in the present invention can include, for example, periodate-oxidized O-desulfated heparin, decarboxylated O-desulfated heparin, acetylated O-desulfated heparin, deacetylated O-desulfated heparin, deacetylated, oxidized O-desulfated heparin, and low molecular weight O-desulfated heparin. Heparin that is 2-O, 3-O desulfated with an average molecular weight of about 4,000 to 12,500 Da is particularly useful in the present invention for treating or preventing ischemia-related intracellular Ca⁺⁺ overload from reverse mode operation of the NCE.

The O-desulfated heparin used according to the present invention can be in any form useful for delivery to a patient provided the O-desulfated heparin maintains the activity useful in the methods of the invention, particularly the low anticoagulation activity of the O-desulfated heparin. Non-limiting examples of further forms the O-desulfated heparin may take on that are encompassed by the invention include esters, amides, salts, solvates, prodrugs, or metabolites. Such further forms may be prepared according to methods generally known in the art, such as, for example, those methods described by J. March, Advanced Organic Chemistry: Reactions, Mechanisms and Structure, 4^{th} Ed. (New York: Wiley-Interscience, 1992), which is incorporated herein by reference.

In the case of solid compositions, it is understood that the compounds used in the methods of the invention may exist in different forms. For example, the compounds may exist in stable and metastable crystalline forms and isotropic and amorphous forms, all of which are intended to be within the scope of the present invention.

### IV. Pharmaceutical Compositions

While it is possible for the O-desulfated heparin used in the methods of the present invention to be administered in the raw chemical form, it is preferred for the compounds to be delivered as a pharmaceutical composition. Accordingly, there are provided by the present invention pharmaceutical compositions including O-desulfated heparin. As such, the compositions used in the methods of the present invention include O-desulfated heparin or pharmaceutically acceptable variants thereof.

The O-desulfated heparin can be prepared and delivered together with one or more pharmaceutically acceptable carriers therefore, and optionally, other therapeutic ingredients. Carriers should be acceptable in that they are compatible with any other ingredients of the composition and not harmful to the recipient thereof. Such carriers are known in the art. See, Wang et al. (1980) J. Parent. Drug Assn. 34(6):452-462, herein incorporated by reference in its entirety.

Compositions may include short-term, rapid-onset, rapid-offset, controlled release, sustained release, delayed release, and pulsatile release compositions, providing the compositions achieve administration of a compound as described herein. See Remington's Pharmaceutical Sciences (18^{th} ed.; Mack Publishing Company, Eaton, Pa., 1990), herein incorporated by reference in its entirety.

Pharmaceutical compositions for use in the methods of the invention are suitable for various modes of delivery, including oral, parenteral, and topical (including dermal, buccal, and sublingual) administration. Administration can also be via nasal spray, surgical implant, internal surgical paint, infusion pump, or other delivery device. The most useful and/or beneficial mode of administration can vary, especially depending upon the condition of the recipient.

In preferred embodiments, the compositions of the invention are administered intravenously, subcutaneously or by direct intra-arterial injection Particularly preferred modes of delivery include parenteral infusions (such as intravenous and subcutaneous infusions) or periodic injections (including intravenous and subcutaneous periodic injections from once up to four times daily). To obtain prompt effects and reduce Ca⁺⁺ overload of tissues immediately after relief of ischemia in an organ, O-desulfated heparin can also be administered as a direct intra-arterial injection into the coronary artery, carotid artery or main aorta at the time blood flow is restored. Alternately, intra-arterial administration of O-desulfated heparin can be slightly delayed until performance of a post-conditioning protocol by repeated short cycles of occlusion followed by restoration of blood flow in the arterial vessel.

The pharmaceutical compositions may be conveniently made available in a unit dosage form, whereby such compositions may be prepared by any of the methods generally known in the pharmaceutical arts. Generally speaking, such methods of preparation include combining (by various methods) the O-desulfated heparin with a suitable carrier or other adjuvant, which may consist of one or more ingredients. The combination of the O-desulfated heparin with the one or more adjuvants is then physically treated to present the composition in a suitable form for delivery (e.g., forming an aqueous suspension).

Compositions for parenteral administration include aqueous and non-aqueous sterile injection solutions, which may further contain additional agents, such as anti-oxidants, buffers, bacteriostats, and solutes, which render the compositions isotonic with the blood of the intended recipient. The compositions may include aqueous and non-aqueous sterile suspensions, which contain suspending agents and thickening agents. Such compositions for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and the like.

In specific embodiments, a patient suffering ischemia can be treated with 2-O, 3-O desulfated heparin produced according to methods outlined in U.S. Pat. No. 5,990,097, which is incorporated herein by reference. In certain embodiments, treatment can be effected by administering an intravenous bolus having an O-desulfated heparin. Such composition can be formed according to various pharmaceutical methods, as discussed herein. Preferably, the bolus is isotonic and has a pH that is neutral to slightly acidic. In a specific embodiment, an intravenous bolus for administration to a patient suffering ischemia includes a 50 mg/ml formulation of 2-O, 3-O desulfated heparin in water with sufficient NaCl added to make the solution isotonic at about 260 to 320 mOsm/ml. The formulation preferably has a pH of about 5 to 7.5. This formulation can be packaged (such as in sterile 20 ml glass vials) and stored at room temperature under low light conditions. Of course, other solution concentrations may also be used, and a skilled person would recognize a suitable concentration for achieving the desired delivery of ODSH in the desired amount of time. For example, an intravenous bolus may include ODSH in a range of about 5 mg/ml to about 100 mg/ml, about 10 mg/ml to about 90 mg/ml, or about 25 mg/ml to about 250 mg/ml.

In one embodiment, a patient is treated by administering a first intravenous bolus of ODSH at doses ranging from 4 to 16 mg/kg, the drug being dissolved in 50 to 100 ml of 5% dextrose in water or 0.9% NaCl. This bolus dose can be followed by a constantly infused dose for up to 96 hours. In specific embodiments, the constantly infused dose is in the range of 1.5 to 2.5 mg/kg/hr. The infused drug can also be diluted in 5% dextrose in water or 0.9% NaCl for infusion.

When treating a patient suffering from ischemia using such a method, the amount of ODSH being used in the bolus and the composition for infusion can vary. For example, the bolus can include ODSH in an amount of about 1.0 mg/kg of patient body weight to about 20 mg/kg of patient body weight. In further embodiments, the bolus can include ODSH in an amount of about 4 mg/kg to about 18 mg/kg, about 4 mg/kg to about 16 mg/kg, about 4 mg/kg to about 12 mg/kg, or about 4 mg/kg to about 8 mg/kg.

In other embodiments, the constantly infused dose can include ODSH in an amount providing for delivery of about 0.05 mg per kg of body weight per hour of delivery (mg/kg/hr) to about 5 mg/kg/hr. In still further embodiments, ODSH can be constantly infused at a rate of about 0.5 mg/kg/hr to about 4 mg/kg/hr, about 0.6 mg/kg/hr to about 3 mg/kg/hr, about 0.8 mg/kg/hr to about 2.5 mg/kg/hr, or about 1.0 mg/kg/hr to about 2.0 Likewise, the duration of the constant infusion can also vary. For example, the constant infusion can be carried out for a time of up to about 168 hours. In further embodiments, the constant infusion can be carried out for a time of about 1 hour to about 168 hours, about 12 hours to about 144 hours, about 24 hours to about 120 hours, about 36 hours to about 96 hours, about 48 hours to about 96 hours, or about 4 hours to about 12 hours. Of course, the duration of the constant infusion may vary based on the concentration of the ODSH in the infused formulation. It is also understood that the treatment by constant infusion as described herein can be carried out in combination with administration of a bolus, as disclosed above, or as a stand-alone treatment (i.e., carried out without prior administration of a bolus dose. Preferably, constant infusion is carried out for a time sufficient to prevent or reduce the Ca⁺⁺ overload within cells resulting from the ischemic condition. In certain embodiments (although not required according to the invention), a patient receiving a constant infusion of ODSH is hospitalized for the ischemic condition. In such embodiments, it is preferable that the constant infusion be carried out until the ischemic injury to the organ or whole individual has been reduced or eliminated such that the patient is discharged from the hospital or can at least be transitioned to oral medications for the ischemia.

The following non-limiting embodiment illustrates the treatment of a patient suffering from ischemia by administering a bolus of 8 mg/kg followed by infusion of 2.0 mg/kg/hr for 24 hours. For each bolus dose, a total of 50 mL of solution can be infused. In order to provide additional solution for priming infusion lines, a total of 75 L can be prepared. For example, for a 70 kg subject receiving a bolus does of 8 mg/kg, Table 1 describes the amount of 2-O, 3-O desulfated heparin (referred to as ODSH), the diluent required, and the final solution concentrations for one exemplary bolus dosing, assuming a stock solution of ODSH of 20 ml bottles containing 50 mg ODSH per ml of solution.

TABLE 1

| Parameter | Amount | |
|---|---|---|
| Infusion bag volume | 100 | (mL) |
| Delivered volume | 50 | (mL) |
| Total prepared volume | 75 | (mL) |
| Patient weight | 70 | (kg) |
| ODSH bolus dose | 8.0 | (mg/kg) |
| Infusion rate | 200 | (mL/hr) |
| Concentration delivered | 11.2 | (mg/mL) |
| Total volume ODSH added to bag | 16.8 | (mL) |
| Total volume saline added to bag | 58.2 | (mL) |

Table 2 illustrates further exemplary formulations for bolus dosing based on patient weight. The bolus doses can be prepared by combining the calculated amounts of 2-O, 3-O desulfated heparin and 0.9% sodium chloride (i.e., normal saline), or other suitable infusion medium, in a sterile infusion bag. An intravenous infusion line can then be attached to the infusion bag, and the infusion set primed with solution. A Luer lock can be placed at the end of the set. Because 2-O, 3-O desulfated heparin doses are weight based, the amount of 2-O, 3-O desulfated heparin and diluent will both vary by subject weight. The examples of Table 3 are based on an infusion bag volume of 100 mL, a delivered volume of 50 mL, a total prepared volume of 75 mL, a bolus dose of 8 mg/kg, an infusion rate of 200 mL/hr, and an infusion duration of 0.25 hours.

TABLE 2

| Body Weight | ODSH Volume | Saline Volume | ODSH Conc. | Dose |
|---|---|---|---|---|
| (kg) | (mL) | (mL) | (mg/mL) | (mg/kg) |
| 45.0 | 10.8 | 64.2 | 7.20 | 8.0 |
| 47.5 | 11.4 | 63.6 | 7.60 | 8.0 |
| 50.0 | 12.0 | 63.0 | 8.00 | 8.0 |
| 52.5 | 12.6 | 62.4 | 8.40 | 8.0 |
| 55.0 | 13.2 | 61.8 | 8.80 | 8.0 |
| 57.5 | 13.8 | 61.2 | 9.20 | 8.0 |
| 60.0 | 14.4 | 60.6 | 9.60 | 8.0 |
| 62.5 | 15.0 | 60.0 | 10.00 | 8.0 |
| 65.0 | 15.6 | 59.4 | 10.40 | 8.0 |
| 67.5 | 16.2 | 58.8 | 10.80 | 8.0 |
| 70.0 | 16.8 | 58.2 | 11.20 | 8.0 |
| 72.5 | 17.4 | 57.6 | 11.60 | 8.0 |
| 75.0 | 18.0 | 57.0 | 12.00 | 8.0 |
| 77.5 | 18.6 | 56.4 | 12.40 | 8.0 |
| 80.0 | 19.2 | 55.8 | 12.80 | 8.0 |
| 82.5 | 19.8 | 55.2 | 13.20 | 8.0 |
| 85.0 | 20.4 | 54.6 | 13.60 | 8.0 |
| 87.5 | 21.0 | 54.0 | 14.00 | 8.0 |
| 90.0 | 21.6 | 53.4 | 14.40 | 8.0 |
| 92.5 | 22.2 | 52.8 | 14.80 | 8.0 |
| 95.0 | 22.8 | 52.2 | 15.20 | 8.0 |
| 97.5 | 23.4 | 51.6 | 15.60 | 8.0 |
| 100.0 | 24.0 | 51.0 | 16.00 | 8.0 |
| 102.5 | 24.6 | 50.4 | 16.40 | 8.0 |
| 105.0 | 25.2 | 49.8 | 16.80 | 8.0 |
| 107.5 | 25.8 | 49.2 | 17.20 | 8.0 |
| 110.0 | 26.4 | 48.6 | 17.60 | 8.0 |
| 112.5 | 27.0 | 48.0 | 18.00 | 8.0 |
| 115.0 | 27.6 | 47.4 | 18.40 | 8.0 |
| 117.5 | 28.2 | 46.8 | 18.80 | 8.0 |
| 120.0 | 28.8 | 46.2 | 19.20 | 8.0 |
| 122.5 | 29.4 | 45.6 | 19.60 | 8.0 |
| 125.0 | 30.0 | 45.0 | 20.00 | 8.0 |
| 127.5 | 30.6 | 44.4 | 20.40 | 8.0 |
| 130.0 | 31.2 | 43.8 | 20.80 | 8.0 |

For each continuous infusion dose, in certain embodiments, a total of 300 mL of diluted ODSH can be prepared. The initial infusion rate can be 10 mL/hr, and the infusion rate may change depending upon activated partial thromboplastin (aPTT) values. For each subject with ischemia, continuous infusions can be prepared at a concentration based upon patient body weight (i.e., the body weight measured within 36 hours of infusion start). Infusion lines are preferentially primed with active drug product. Preferentially, the ODSH is maintained in refrigerated conditions (e.g., in the range of 2-8° C.) until used. The infusion solution should be allowed to reach room temperature prior to administration. For example, for a 70 kg subject receiving a continuous infusion of 2.0 mg/kg/hr, Table 3 below describes the amount of ODSH and saline required, as well as the final solution concentration, for each 24 hour infusion period.

TABLE 3

| Parameter | Amount | |
|---|---|---|
| Delivered volume | 240 | (mL) |
| Total prepared volume | 300 | (mL) |
| Patient weight | 70 | (kg) |
| ODSH dose | 48 | (mg/kg/24 hr) |
| ODSH dose | 2.0 | (mg/kg/hr) |
| ODSH dose | 3,360 | (mg/24 hr) |
| Infusion rate | 10 | (mL/hr) |
| Volume of saline added to bag | 216 | (mL) |
| Volume ODSH delivered in 24 hr | 240 | (mL) |
| Concentration delivered | 14 | (mg/mL) |
| Total volume ODSH (50 mg/ml | 84 | (mL) |
| stock solution) added to bag | | |

In other embodiments, similar calculations may be required to make up solution bags for individuals based on different weights so that ODSH may be provided at an infusion rate of 10 ml/ml by infusion pump for 24 hours, resulting in accurate delivery of 2 mg/kg/hr. For example, a bolus of 8 mg/kg followed by 2 mg/kg/hr would be predicted to give an ODSH blood concentration of approximately 100 μg/mL. This concentration would provide maximal possible inhibition of injurious intracellular Ca⁺⁺ accumulation in the ischemic organ of reference. This concentration would also predictably increase the aPTT to about 50 seconds above baseline, or for a baseline of 24 seconds, to an absolute aPTT value of 75 seconds, which is in the range of therapeutic clinical anticoagulation. The infusion rate of ODSH may be increased or decreased as needed to titrate to a therapeutic aPTT range of between 60 to 80 seconds, with monitoring of the aPTT beginning 6 hours after the ODSH bolus, and again at 12 to 24 hour intervals. This ODSH regimen of a bolus of 8 mg/kg followed by 2 mg/kg/hr for 24 hours may be provided to subjects experiencing cardiac ischemia treated with thrombolytic agents. For example, an individual may be first bolused with ODSH at 8 mg/kg and started on an ODSH infusion at 2 mg/kg/hr. The subject may then be treated with intravenous streptokinase, tissue plasminogen activator, reteplase or tenecteplase employing usual clinical protocols. Blood levels of ODSH achieved in this embodiment would predictably provide ODSH concentrations of approximately 100 μg/mL, effectively inhibiting injurious intracellular Ca⁺⁺ accumulation once restoration of blood flow to the ischemic myocardium was effected by action of the thrombolytic agent.

A well-known side effect of thrombolytic agents is central nervous system hemorrhage occurring in 0.5 to 3.0% of individuals treated with these agents. Because of this unpredictable side effect, a safer mode of treatment for the individual suffering cardiac ischemia from coronary occlusion would be to proceed to immediate emergency cardiac catheterization with rescue angioplasty and stent placement to relief coronary occlusion. In this clinical situation, it is customary to anticoagulate the patient with heparin, low molecular weight heparin or a direct thrombin inhibitor to prevent clot formation on the cardiac catheters as they are inserted into the arterial system. When heparin is used, sufficient heparin is injected to elevate the activated clotting time (ACT) test to between 200 and 250 seconds, with additional heparin boluses to keep the ACT within the range, thereby preventing clot formation on the cardiac catheters. The normal range for ACT values in unanticoagulated subjects varies from laboratory to laboratory, but ranges from 100 to 150 seconds. In one embodiment, in order to place the ACT immediately within the therapeutic anticoagulation target for cardiac catheterization of 200 to 250 seconds, the treating physician can administer a bolus dose of 16 mg/kg ODSH, or twice the previously described bolus. For example, a 70 kg subject, this can be done by infusing 50 ml of a 100 ml bolus infusion bag over 15 minutes, preparing the infusion bag for the 70 kg adult according to Table 4:

TABLE 4

| Parameter | Amount | |
|---|---|---|
| Infusion bag volume | 100 | (mL) |
| Delivered volume | 50 | (mL) |
| Total prepared volume | 75 | (mL) |
| Patient weight | 70 | (kg) |
| ODSH bolus dose | 16.0 | (mg/kg) |
| Infusion rate | 200 | (mL/hr) |
| Concentration delivered | 22.4 | (mg/mL) |
| Total volume ODSH added to bag | 33.6 | (mL) |
| Total volume saline added to bag | 41.4 | (mL) |

The subject suffering ischemia can then be periodically bolused second, third or fourth times with 16 mg/kg at intervals to maintain the ACT in the range of 200 to 250 seconds, or preferably, he can be started on a constant infusion of ODSH at 2 mg/kg/hr according to the directions outlined above in Table 3 for a subject weighing 70 kg. The infusion can then be continued for 12 to 24 hours to prevent or reduce injurious intracellular Ca⁺⁺ accumulation for this period. In certain aspects, the advantage of bolus ODSH is that the blood contains approximately 100 μg/mL or more of ODSH so that injurious intracellular Ca⁺⁺ accumulation is maximally inhibited when blood flow is restored to the ischemic myocardium with dissolution of clot within the coronary. Used in this manner, ODSH can also be combined with an ischemic post-conditioning protocol, in which brief one-minute periods of occlusion followed by one-minute periods of reflow are performed for four to six times in the coronary by alternate inflation and deflation of the angioplasty catheter following deployment of the coronary stent. In this manner the benefit of ischemic post-conditioning as previously discussed can be combined with the benefit of reducing injurious intracellular Ca⁺⁺ accumulation through application of ODSH. In one embodiment, at the end of stent deployment (if a post-ischemic conditioning protocol is not employed) or at the end of the ischemic post-conditioning protocol, 5 to 100 mg of ODSH (0.1 to 2.0 mL of 50 mg/mL stock solution or the same concentration of ODSH diluted in a higher volume with saline) can be injected directly into the previously ischemic coronary to provide immediate delivery of inhibitory doses of ODSH to prevent injurious intracellular Ca⁺⁺ accumulation within the ischemic myocardial bed. Direct coronary inject of heparin has been previously described and direct coronary injection of these amounts of ODSH will be not only safe but beneficial to the patient's recovery with minimal myocardial injury from ischemia.

In one embodiment, in order to prevent injurious intracellular Ca⁺⁺ accumulation from with Ca⁺⁺ overload at the time of cardiopulmonary bypass for heart surgery, ODSH may be utilized instead of heparin to anticoagulate the patient during cardiopulmonary bypass. For example, doses similar to the 16 mg/kg bolus and 2-4 mg/kg/hr infusion may be required to maintain the ACT in a desired therapeutic range. Alternatively, anticoagulation with heparin can be employed and ODSH can be injected directly into the coronary arteries by a rapid infusion into the aortic arch of 50 to 250 mg ODSH diluted in saline just prior to the end of cardioplegic arrest to provide high concentrations of ODSH in the early myocardial blood flow and prevent or reduce injurious intracellular Ca⁺⁺ accumulation as cardioplegia is ended and the heart is defibrillated. If ODSH is used instead of heparin as the anticoagulant, its anticoagulant activity can be reversed by protamine injections at the end of bypass, just as is currently done with heparin, which provides a safe reduction in the level of anticoagulation to prevent bleeding into the mediastinum as bypass is discontinued.

One common cause of whole body ischemia leading to dangerous intracellular Ca⁺⁺ overload is cardiopulmonary arrest, a condition in which the heart effectively stops pumping blood to vital organs because of the development of ineffective rhythms such as ventricular tachycardia or ventricular fibrillation. In this case, ischemia in all vital organs produces intracellular Na⁺ accumulation so that reverse mode operation of the NCE produces widespread Ca⁺⁺ overload within many organs if or when normal cardiac rhythm is restored with cardiopulmonary resuscitation and defibrillation. The consequences of widespread Ca⁺⁺ overload include anoxic encephalopathy, in which necrosis and apoptosis of the ischemic brain produces coma or serious loss in mental function despite adequate restoration of cardiac performance. Other conditions accompanying the widespread Ca⁺⁺ overload from cardiopulmonary arrest include hepatic injury, often termed “shock liver”, ischemic bowel necrosis, often termed “ischemic colitis”, and renal injury, often termed “acute renal failure” or “acute renal tubular necrosis.” In one embodiment, these conditions can be prevented and reduced, and the restoration of normal cardiac rhythm can be restored by the injection of about 8 to about 16 mg/kg ODSH into the venous circulation of an individual suffering cardiopulmonary arrest at the earliest point when intravenous access is available during the resuscitative effort. In this embodiment, ODSH can then be continued as a constant infusion at rates of about 1.0 to about 2.0 mg/kg/hr to provide a continuous level of drug for up to 12 hours to reduce or prevent widespread Ca⁺⁺ overload accompanying the return of adequate cardiac output.

Treatment of central nervous system ischemia from arterial occlusion from in situ thrombosis or embolic obstruction requires modification of the above protocols because of the peculiar risk of hemorrhage within the brain substance if anticoagulation is present in the early days after relief of brain ischemia. Presently brain ischemia is treated in a few cases by intravenous administration of tissue plasminogen activator. As interventional neuro-radiologists become more aggressive in their therapy of arterial occlusions, patients will in the future be able to experience mechanical disruption of clot occluding the cerebral vasculature just as readily as patients do who are treated in such a fashion as therapy for cardiac ischemia. In such situation, when cerebral vascular occlusion is relieved by direct mechanical disruption of the occlusion, an ischemic post-conditioning protocol for the central nervous system similar to that described for the cardiac system can be employed to decrease cerebral injury. In one embodiment, ODSH in doses of about 5 to about 250 mg can be injected directly into the occluded cerebral vessel at the time occlusion is relieved, or immediately following performance of a post-ischemic conditioning protocol. Administered in this manner, ODSH will prevent or reduce widespread Ca⁺⁺ overload in ischemic cerebral tissue. This treatment algorithm can reduce ischemic cerebral injury.

To prevent widespread Ca⁺⁺ overload from ischemic injury to the lower body as a consequence of surgery for treatment of aortic aneurysm, ODSH can be used in a manner described above to anticoagulate the patient instead of heparin. As outlined, when anticoagulation is reduced at the end of the surgical procedure, the level of anticoagulation from ODSH can be reduced by protamine injections in a fashion similar to that followed to reduce the level of anticoagulation from heparin. In addition to surgery for aortic aneurysm, ODSH can be useful when employed instead of heparin for medical and/or surgical treatment of ischemic lower extremities to prevent tissue loss and destruction consequent to widespread Ca⁺⁺ overload from disruption of blood flow to the legs.

In certain embodiments, treatment of a patient with ischemia using a bolus dose of 2-O, 3-O desulfated heparin followed by a constant infusion dose is particularly beneficial in that it will not cause a fall in platelets. In certain embodiments, the ODSH treatment can be administered in conjunction with anti-platelet agents, oxygen, antibiotics, corticosteroids, vasopressors, anti-arrhythmic agents, beta-blocking agents, and, if needed non-invasive or mechanical ventilation. In most subjects treated with these doses in this manner along with conventional therapy, the patient will experience sufficient improvement in the ischemic symptoms and consequences of widespread Ca⁺⁺ overload to experience 10 to 50% reduction in the amount of organ dysfunction that would otherwise result from ischemic insult.

In another embodiment, desulfated heparin can be administered subcutaneously. With such administration, the drug may be formulated in concentrations suitable for subcutaneous administration. For example, in certain embodiments, a formulation for subcutaneous administration can include ODSH in a concentration of about 5 mg/ml to about 500 mg/ml, about 10 mg/ml to about 450 mg/ml, about 15 mg/ml to about 400 mg/ml, about 20 mg/ml to about 350 mg/ml, about 25 mg/ml to about 325 mg/ml, about 30 mg/ml to about 300 mg/ml, about 35 mg/ml to about 275 mg/ml, about 40 mg/ml to about 250 mg/ml, about 45 mg/ml to about 225 mg/ml, or about 50 mg/ml to about 200 mg/ml.

The desired amount of ODSH can be combined with a suitable medium such as, for example, isotonic saline or sterile water, and injected via the desired method. For example, the formulation may be injected periodically in volumes up to about 2.0 mL subcutaneously.

Alternatively, the formulation can be constantly infused into the subcutaneous space by a small gauge butterfly needle (e.g., a 21 to 23 gauge needle). In still further embodiments, a subcutaneous soft catheter of the variety used for insulin infusion can be used to constantly infuse drug subcutaneously. This catheter is conveniently placed into the subcutaneous space of the anterior abdominal wall. A particularly useful catheter for this purpose is the SOF-SET QR®, which can be purchased from the Medtronic Corporation in Northridge, Calif. This catheter is particularly advantageous because it allows for self-placement by patients.

In one embodiment, once the catheter or butterfly needle is inserted, the patient can receive a constant infusion of drug by loading an appropriate amount of a formulation (e.g., about 50 mg/mL) into a syringe. The syringe is then placed into the carriage of a mechanical infusion pump, such as the FREEDOM60® infusion pump available from RMS Medical Products in Chester, N.Y. Connected to an indwelling subcutaneous infusion catheter, this pump-catheter infusion system will infuse O-desulfated heparin at a stable, constant rate for up to 72 hours at infusion rates as high as 0.55 mg/kg/hr.

Alternatively, the drug formulation can be diluted similarly to that outlined above for continuous intravenous infusion and administered by continuous subcutaneous infusion using a CADD® infusion pump manufactured by Smith Medical International, Colonial Way, Watford, UK.

In certain embodiments, the compounds and compositions disclosed herein can be delivered via a medical device. Such delivery can generally be via any insertable or implantable medical device including, but not limited to, stents, catheters, balloon catheters, shunts, or coils. In one embodiment, the present invention provides medical device such as, for example, a stent, where the surface of the stent is coated with a compound or composition as described herein. The medical device of this invention can be used, for example, in any application for treating, preventing, or otherwise affecting the course of a disease or condition, such as those disclosed herein.

In another embodiment of the invention, pharmaceutical compositions composed of O-desulfated heparin can be administered intermittently. Administration of the therapeutically effective dose may be achieved in a continuous manner, as for example with a sustained-release composition, or it may be achieved according to a desired daily dosage regimen, as for example with one, two, three, or more administrations per day. The phrase “time period of discontinuance”” is defined herein as the period when no compound is administered to the subject. The time period of discontinuance may be longer or shorter than the period of continuous sustained-release or daily administration. During the time period of discontinuance, the level of the components of the composition in the relevant tissue is substantially below the maximum level obtained during the treatment. The preferred length of the discontinuance period depends on the concentration of the effective dose and the form of composition used. The discontinuance period can be at least 2 days, at least 4 days or at least 1 week. In other embodiments, the period of discontinuance is at least 1 month, 2 months, 3 months, 4 months or greater. When a sustained-release composition is used, the discontinuance period must be extended to account for the greater residence time of the composition in the body. Alternatively, the frequency of administration of the effective dose of the sustained-release composition can be decreased accordingly. An intermittent schedule of administration of a composition of the invention can continue until the desired therapeutic effect, and ultimately treatment of the disease or disorder, is achieved.

Administration of the composition can include administering O-desulfated heparin in combination with one or more pharmaceutically active agents (i.e., co-administration). Accordingly, it is recognized that the pharmaceutically active agents described herein can be administered in a fixed combination (i.e., a single pharmaceutical composition that contains both active agents). Alternatively, the pharmaceutically active agents may be administered simultaneously (i.e., separate compositions administered at the same time). In another embodiment, the pharmaceutically active agents are administered sequentially (i.e., administration of one or more pharmaceutically active agents followed by separate administration or one or more pharmaceutically active agents). One of skill in the art will recognize that the most preferred method of administration will allow the desired therapeutic effect.

Delivery of a therapeutically effective amount of a composition according to the invention may be obtained via administration of a therapeutically effective dose of the composition. Accordingly, in one embodiment, a therapeutically effective amount is an amount effective to reduce or maintain intracellular Ca⁺⁺ levels during an ischemic event or prevent an increase in intracellular Ca⁺⁺ levels prior to an ischemic event. In another embodiment, a therapeutically effective amount is an amount effective to treat a symptom of ischemia. In yet another embodiment, a therapeutically effective amount is an amount effective to prevent the onset of a symptom associated with ischemia.

The concentration of O-desulfated heparin in the composition will depend on absorption, inactivation, and excretion rates of the O-desulfated heparin as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

It is contemplated that compositions of the invention including one or more active agents described herein can be administered in therapeutically effective amounts to a mammal, preferably a human. An effective dose of a compound or composition for treatment of any of the conditions or diseases described herein can be readily determined by the use of conventional techniques and by observing results obtained under analogous circumstances. The effective amount of the compositions would be expected to vary according to the weight, sex, age, and medical history of the subject. Of course, other factors may also influence the effective amount of the composition to be delivered, including, but not limited to, the specific disease involved, the degree of involvement or the severity of the disease, the response of the individual patient, the particular compound administered, the mode of administration, the bioavailability characteristics of the preparation administered, the dose regimen selected, and the use of concomitant medication. The compound is preferentially administered for a sufficient time period to alleviate the undesired symptoms and the clinical signs associated with the condition being treated. Methods to determine efficacy and dosage are known to those skilled in the art. See, for example, Isselbacher et al. (1996) Harrison's Principles of Internal Medicine 13 ed., 1814-1882, herein incorporated by reference.

In certain embodiments for intravenous administration, a composition of the invention can be dosed at about 4-20 mg/kg of bodyweight and infused at a rate of about 0.5 to about 2.5 mg/kg/hr. For subcutaneous administration, a patient can be given an initial dose of about 4-16 mg/kg followed by doses of about 6-18 mg/kg subcutaneously every 24 hours in at least two divided doses. Of course, the above dosages are intended from purposes of guidance and are not intended to limit the scope of the invention.

In other specific embodiments, treatment bolus doses can range from about 2.0 mg/kg to about 20.0 mg/kg administered intravenously over about 15 minutes or even subcutaneously over about 30 minutes. Constant infusion doses administered intravenously or subcutaneously range from about 0.5 mg/kg/hr to about 4.0 mg/kg/hr for up to about 48 hours. Periodic intravenous or subcutaneous injection doses can include a total of about 8 mg/kg to about 16 mg/kg administered intravenously or subcutaneously. The doses can be administered every 24 hours in two to four divided doses for up to 4 days.

### EXPERIMENTAL

The present invention will now be described with specific reference to various examples. The following examples are not intended to be limiting of the invention and are rather provided as exemplary embodiments.

### Example 1

### Inhibition of Ischemia-Induced Intracellular Calcium Overload by 2-O, 3-O Desulfated Heparin

This example demonstrates how 2-O, 3-O desulfated heparin prevents dangerous accumulation of intracellular calcium in cardiac myocytes by blocking ischemia-induced increases in the late sodium current. Consequently, it prevents intracellular sodium overload and subsequent reverse mode operation of the sodium-calcium exchanger. The effect of 2-O, 3-O desulfated heparin on accumulation of intracellular Ca⁺⁺ during ischemia was studied in an adult rabbit ventricular myocyte model previously reported (Boston D R, et al. J Pharmacol Exp Ther 285:716-723, 1998; Li F, et al. J Mol Cell Cardiol 33:2145-2155, 2001). Isolated cardiomyocytes have been validated as reliable models of ischemia (Diaz, R J, Wilson G J. Cardiovasc Res 70:286-296, 2006).

To produce the model, hearts are removed from albino rabbits (2-3 kg) anesthetized with sodium pentobarbital (65 mg/kg IV). The heart was immediately attached to an aorta cannula. Continuous perfusion of the coronary arteries at 37° C. by a pump (Masterflex, Cole-Parmer Instrument Co., Chicago, Ill.) was initiated at a perfusion pressure of 60 mm Hg. The heart was first perfused with nominally Ca⁺⁺-free modified Krebs-Ringer bicarbonate buffered solution (MKRBB, pH 7.40, containing in mmol/L: NaCl 126, KCl 4.4, CaCl₂ 1.08, MgCl₂ 1.0, HEPES 24, probenecid 0.5 and glucose 5) for 10 min, immediately followed by 15-25 min of recirculating perfusion with the same solutions containing 0.3 mg/ml collagenase (Type 2, Worthington Biochemical, Freehold, N.J.), 0.4 mg/mL hyaluronidase (Type-S, Sigma-Aldrich, St. Louis, Mo.) and 50 μmol/L CaCl₂. The heart was then detached from the cannula, and the left ventricle was minced and transferred to a 50 mL conical tube with the same solution containing 0.24 mg/mL collagenase, 0.02 mg/mL trypsin and 50 μmol/L Ca⁺⁺ for a second digestion. The minced tissue was continuously agitated by gassing the solution with 5% CO₂ and 95% O₂ to help release isolated myocytes. The resulting supernatant was transferred to another conical tube with the same volume of the same solution containing 0.03 mg/mL of trypsin inhibitor (Sigma-Aldrich). The cell suspension was centrifuged at 5×g for 5 minutes. The supernatant was discarded and cells were resuspended in solution with a higher Ca⁺⁺ concentration (200 μmol/L) and incubated in a CO₂ incubator (5% CO₂, 28° C.) to settle down cells by gravity for 10 min. The same procedure was repeated twice to bring up the Ca⁺⁺ concentration slowly (500, then 1000 μmol/L). Calcium step-up solutions were made from MKRBB with 2% albumin, 50 μmol/L CaCl₂ mixed with the appropriate amount of minimum essential medium (MEM, Gibco Laboratories, Grand Island, N.Y.). At the end of isolation, cells were filtered with a 300 μmol/L filter to avoid cell clumping during flow cytometry studies. The yield of rod-shaped viable myocytes averaged 50%. Cells were used for experiments within 4 hours after dissociation.

In these studies, intracellular calcium concentration [Ca⁺⁺]ᵢ was measured by flow cytometry. Measurement of isolated mouse and rabbit ventricular myocyte [Ca²⁺]ᵢ and [Na⁺]ᵢ by flow cytometry has been described (Zhang et al., J. Cardiovasc. Pharmacol. 51: 443-449 (2008); Li et al., J. Mol. Cell. Cardiol. 33: 2145-2155 (2001)). [Ca⁺⁺]ᵢ was measured with the Ca⁺⁺ sensitive fluorescent probe, Fluo 3-AM (Molecular Probes, Eugene, Oreg.), using a FACScan (Becton-Dickinson, MA). The cells were exposed to 4 μmol/L Fluo-3 AM for 30 min at 37° C. The cells were then separated into aliquots. After 40 min of wash, the cells were exposed to paced metabolic ischemia (MI) for 45 min at 37° C. Metabolic ischemia was accomplished by incubating cells in a glucose-free MKRBB solution (pH 7.40) containing 2 mmol/L sodium cyanide (NaCN) to impair mitochondrial and glycolytic generation of ATP. To reproduce the situation in the beating ischemic heart, during the period of metabolic ischemia, myocytes were placed in a 5-well chamber with equal volumes of myocyte suspensions from the same heart dissociation placed in each well. The chamber was water bathed to maintain temperature at 37° C. and bubbled with 5% CO₂ in air to keep myocytes in suspension. Each well was fitted with platinum sheet electrodes on both sides for field stimulation. One chamber was constructed with a glass coverslip bottom so that myocytes can be observed microscopically to assess capture threshold. Placing electrodes were connected in series to a constant current pulse generator that delivers a 0.2 ampere pulse at 0.5 Hz. After 45 min pacing, a sample from each well was taken for determination of intracellular Ca⁺⁺ concentration by flow cytometry. In this fashion, myocytes were electrically paced during ischemia at a rate of 30 beats per minute to produce paced metabolic ischemia (PMI) At 30-50 seconds prior to data acquisition, 10 μL of 20 μmol/L propidium iodide (PI, Molecular Probes) was added to the solution to identify non-viable myocytes. PI is an impermeant probe that is fluorescent only when bound to DNA and was therefore a marker for non-viability (sarcolemmal disruption). After an appropriate time, a sample from a well was taken for determination of [Ca²⁺]ᵢ as described above. During flow cytometry, cells were excited by an argon laser beam (excitation wavelength, 488 nm). Side and forward scattering characteristics were employed to separate the cells from debris. Approximately 2×10³ myocytes were analyzed to calculate average emission fluorescence intensity in each sample. Data were collected for emission intensity at wavelengths of 530 nm for Fluo-3 and 670 nm for PI and plotted simultaneously. Probenecid 0.5 mmol/L was present during loading, wash and protocol solutions to prevent loss of Fluo-3 via the anion transporter. Only those cells with low fluorescence intensity at 670 nm (PI-negative cells or viable cells) were included in the comparative analysis of [Ca⁺⁺]ᵢ. The sample [Ca²⁺]ᵢ values were calculated by Mn²⁺ quenching (Sugishita et al, 2001) and [Na⁺]ᵢ values by comparison with a standard curve of varied [Na⁺]ᵢ versus Na Green fluorescence. Average values for [Ca⁺⁺]ᵢ were calculated as: [Ca⁺⁺]ᵢ=K_{d}×(F−Fₘᵢₙ)/Fₘₐₓ−F), where K_{d}=dissociation constant (864 at 37° C.); Fₘₐₓ=fluorescence intensity at saturating Ca⁺⁺, was estimated as 5×F_{Mn}; F_{Mn}=fluorescence intensity at saturating Mn⁺⁺); Fₘᵢₙ=fluorescence intensity in the absence of Ca⁺⁺ calculated as 1/40 Fₘₐₓ. F_{Mn} is obtained by exposing myocytes to NaCN for 60 min, then to NaCn solution with MnCl₂ 10 mmol/L for 5-10 min. Results were expressed as means±SEM. A paired t-test was used to assess the difference between two groups. A P value <0.05 was considered significant.

In this model, intracellular Ca⁺⁺ progressively rose during PMI as the consequence of intracellular Na⁺ accumulation, with reverse mode operation of the sodium/calcium exchanger (NCE) to export Na⁺ to the external environment while internalizing Ca⁺⁺. To determine the effect of 2-O, 3-O desulfated heparin (ODSH) on intracellular Ca⁺⁺ accumulation under these circumstances, ODSH in concentrations of 1, 10 and 100 μg/mL was added to some wells along with the CN-containing but glucose-free MKRBB solution at the beginning of PMI conditions.

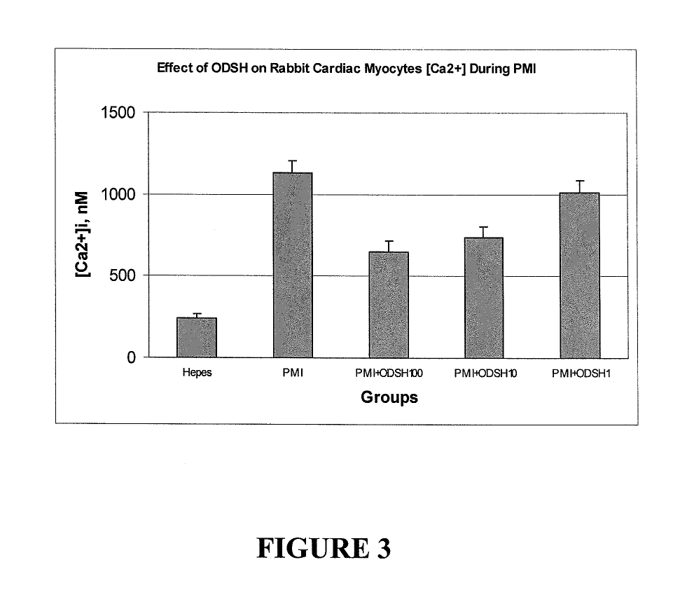

FIG. 3 and Table 5 show the effect of 2-O, 3-O desulfated heparin (ODSH) on intracellular calcium concentration [Ca⁺⁺]_{I} in rabbit ventricular myocytes exposed to normal conditions (Hepes) or conditions of paced metabolic ischemia by culture under glucose-free conditions in a solution containing cyanide to impair mitochondrial and glycolytic generation of ATP(PMI). PMI conditions increase [Ca⁺⁺]ᵢ but addition of ODSH significantly inhibits intracellular Ca⁺⁺ accumulation in a dose dependent manner (P<0.01 vs PMI for 10 μg/mL ODSH+PMI; **<0.001 vs PMI for 100 μg/mL ODSH+PMI).

TABLE 5
Effect of ODSH on Rabbit Cardiac Myocyte [Ca⁺⁺]ᵢ
during Paced Metabolic Ischemia (PMI)

| | [Ca⁺⁺]ᵢ (nM) |
|---|---|
| Groups | (n = 7) |
| Normal Hepes buffer without PMI | 241.9 ± 23.0 |
| PMI | 1133.6 ± 78.4 |
| PMI + ODSH 1 μg/mL | 1013.8 ± 69.2* |
| PMI + ODSH 10 μg/mL | 739.0 ± 63.8** |
| PMI + ODSH 100 μg/mL | 684.3 ± 66.9 |

*P < 0.01 vs PMI without ODSH;
**P < 0.001 vs PMI without ODSH

There is considerable support for the idea that myocyte Ca²⁺ loading via reverse Na⁺/Ca²⁺ exchange (NCX), triggered by increased Na⁺ loading and myocyte depolarization, is an important cause of reperfusion injury. Isolated adult ventricular myocytes subjected to simulated ischemia (metabolic inhibition with CN, and 0 glucose) provide a model in which we have shown Ca²⁺ influx via NCX contributes to Ca²⁺ loading, and in which the degree of rise in [Ca²⁺]ᵢ directly correlates with the degree of injury in the intact heart during ischemia/reperfusion (Zhang et al, 2008). Therefore, the effects of ODSH on [Ca²⁺]ᵢ in this model were examined. FIG. 3 shows the effects of different concentrations of ODSH on myocyte [Ca²⁺]ᵢ during 45 min of simulated ischemia (P-MI). ODSH induced a dose-dependent reduction of [Ca²⁺]_{I}, with a substantial effect at 100 μg/ml, a concentration similar to that present in the serum of humans when therapeutic anticoagulation is achieved during ODSH dose-escalation studies (Phase I safety data on file with FDA for IND #72,247, submitted by ParinGenix, Inc., Weston, Fla.).

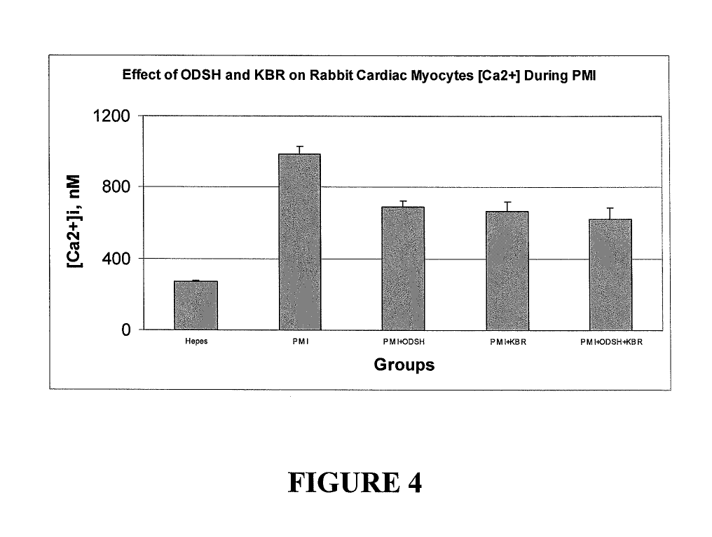

To gain information as to the mechanism by which ODSH inhibits intracellular Ca⁺⁺ accumulation during paced ischemia, separate experiments were performed in which some cells were treated with KB-R7943 (KBR), which inhibits reverse mode operation of the sodium/calcium exchanger (NCE) at a concentration of 10 μmol/L. Specifically, this study was done to determine if Ca²⁺ influx via reverse mode NCX was involved in this effect of ODSH, the reduction in [Ca²⁺]ᵢ induced by ODSH in the presence of the reverse mode NCX inhibitor. FIG. 4 and Table 6 show that ODSH at 100 μg/mL has no additional protective effect against accumulation of Ca⁺⁺ in this model of paced ischemia when KBR is simultaneously present. The results indicate that KBR and ODSH caused a similar reduction in myocyte [Ca²⁺]ᵢ during 45 min of simulated ischemia, and in the presence of KBR, ODSH caused no significant further reduction in [Ca²⁺]ᵢ. These observations suggest that ODSH could be reducing Ca²⁺ loading either by directly inhibiting NCX, or by reducing Na⁺ loading, and thereby indirectly inhibiting reverse mode NCX. In other words, the ODSH might be working in part through a mechanism that prevents reverse mode operation of the NCE or through some other ion channel effect that affects the NCE indirectly.

TABLE 6
Effect of ODSH and KBR on Rabbit Cardiac Myocyte
[Ca⁺⁺]ᵢ during Paced Metabolic Ischemia (PMI)

| | [Ca⁺⁺]ᵢ (nM) |
|---|---|
| Groups | (n = 7) |
| Normal Hepes buffer without PMI | 269.6 ± 8.3 |
| PMI | 985.5 ± 42.7 |
| PMI + ODSH 100 μg/mL | 688.6 ± 33.4* |
| PMI + KBR 10 μmol/L | 666.5 ± 50.7* |
| PMI + ODSH + KBR | 622.5 ± 60.2* |

*P < 0.001 vs PMI without ODSH or KBR

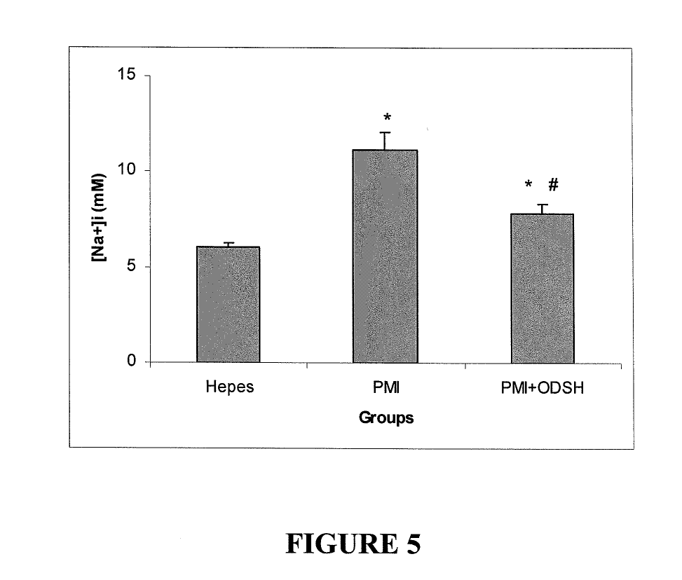

One mechanism that may reduce reverse mode operation of the NCE is blockade of Na⁺ channels by ODSH, thereby preventing an increase of intracellular sodium concentration [Na⁺]ᵢ during ischemia or during rapid augmentation of I_{Na} from burst production of reactive oxygen species during the early minutes after cessation of ischemia and restoration of blood flow. To determine if ODSH hydrocarbyl, substituted hydrocarbyl, heterohydrocarbyl, substituted heterohydrocarbyl, polyether, polyamide, polyimino, aryl, polyester, polythioether, polysaccharyl, or combinations thereof had an effect on Na⁺ channels, myocytes were studied under normal conditions (Hepes) versus during paced metabolic ischemia (PMI) produced as above, but loaded cells with the sodium-sensitive fluorescent probe Sodium Green (Molecular Probes) at a final concentration of 5 μmol/L. Fluorescence was excited in myocytes using an argon laser (excitation wavelength 488 nm) and detected by FACScan at 580 nm. FIG. 5 and Table 7 show that PMI significantly increases [Na⁺]ᵢ in ventricular myocytes. This increase in [Na⁺]ᵢ was significantly reduced by addition of ODSH to the medium at a concentration of 100 μg/mL during PMI. These results suggested that ODSH applied to the external medium blocks Na⁺ channels and I_{Na} in paced ventricular myocytes.

TABLE 7
Effect of ODSH on Rabbit Cardiac Myocyte [Na⁺]ᵢ
during Paced Metabolic Ischemia (PMI)

| | [Na⁺]ᵢ (nM) |
|---|---|
| Groups | (n = 7) |
| Normal Hepes buffer without PMI | 6.04 ± 0.25 |
| PMI | 11.16 ± 0.90* |
| PMI + ODSH 100 μg/mL | 7.85 ± 0.45^{#} |

P < 0.001 vs Hepes;
^{#}P < 0.01 vs PMI

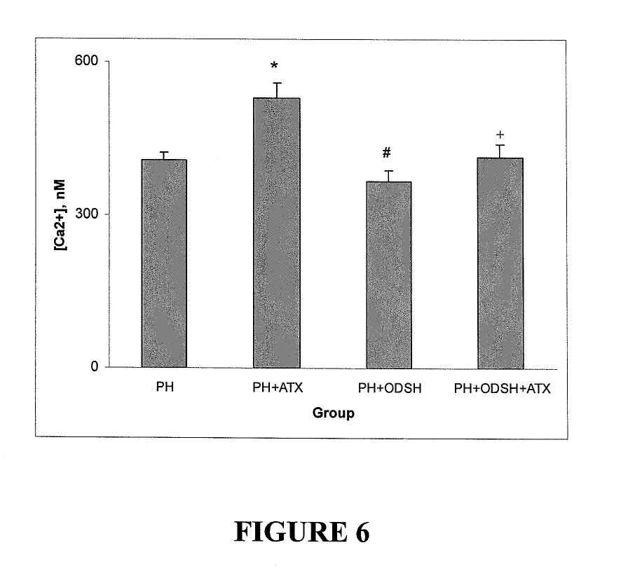

To further examine the role of ODSH as a Na⁺ inhibitor, additional experiments were performed in which the Na⁺ channel opener amenone toxin II (ATX) was added to medium in a final concentration of 20 nmol/L to open the cardiac myocyte membrane Na⁺ channel. Ventricular myocytes were paced as above in Hepes but not exposed to metabolic ischemia (PH). Intracellular calcium concentration [Ca⁺⁺]ᵢ was monitored as earlier. FIG. 6 and Table 8 show that ATX significantly increases [Ca⁺⁺]ᵢ during pacing under aerobic conditions. This effect was produced by the elevation of [Na⁺]ᵢ, with stimulation of reverse mode operation of the NCE to extrude Na⁺ in exchange for Ca⁺⁺, thereby raising [Ca⁺⁺]ᵢ. The addition of 100 μg/mL ODSH to the external medium significantly reduced [Ca⁺⁺]ᵢ compared to PH alone, and prevented the increase in [Ca⁺⁺]ᵢ from the Na⁺ opener ATX applied during PH. This indicated that ODSH applied to the external medium was able to counteract the Na⁺ channel opener ATX by blocking Na⁺ channels. This prevents a rise in [Na⁺]ᵢ, and secondarily an increase in [Ca⁺⁺]ᵢ from reverse mode operation of the NCE.

TABLE 8
Effect of ODSH on Rabbit Cardiac Myocyte [Ca⁺⁺]ᵢ
during Paced Aerobic Metabolism (PH) in the Presence
of the Na⁺ Channel Opener Anemone Toxin II (ATX)

| | [Ca⁺⁺]ᵢ (nM) |
|---|---|
| Groups | (n = 7) |
| Normal Hepes buffer + pacing (PH) | 406.8 ± 15.9 |
| PH + ATX | 529.1 ± 29.3* |
| PH + ODSH 100 μg/mL | 366.4 ± 21.1^{#} |
| PH + ODSH + ATX | 412.9 ± 26.7⁺ |

P < 0.001 vs Hepes;
^{#}P < 0.01 vs PMI;
⁺P < 0.005 vs PH + ATX

The ability of Na⁺ channel inhibitors to prevent an increase in [Na⁺]ᵢ and block reverse mode operation of the NCE with a subsequent rise in [Ca⁺⁺]ᵢ has been recently been demonstrated by treatment of paced ischemic myocytes with the Na⁺ channel inhibitor ranolazine (Barry W, et al. J. Cardiovasc. Cardiol. in press, 2008). In this cited investigation, ventricular myocytes exposed to paced metabolic ischemia (PMI) produced identically as in the examples above experienced a sustained increase in reactive oxygen species production intracellularly, producing a rise in [Na⁺]ᵢ and secondarily [Ca⁺⁺]ᵢ from reverse mode operation of the NCE. These effects of PMI on [Na⁺]ᵢ and [Ca⁺⁺]ᵢ were prevented by treatment of ventricular myocytes with the free radical scavenging drug Tiron, indicating that the augmentation of Na⁺ channels and the late sodium current I_{Na} is directly the result of reactive oxygen species stress and its effects to augment I_{Na}. The addition of the NCE inhibitor KBR preventing a rise in [Ca⁺]ᵢ by blocking reverse mode operation of the NCE as [Na⁺]ᵢ rose. This study demonstrates that oxidative stress increases the late inward sodium current I_{Na} in PMI in a manner similar to the situation when whole heart ischemia is relieved by restoration of blood flow, producing a burst of reactive oxygen species and an increase in I_{Na}. The Na⁺ channel inhibitor ranolazine prevented intracellular Na⁺ and Ca⁺⁺ loading and also blocked hypercontracture of the individual myocytes produced by excessive intracellular Ca⁺⁺. Because ODSH behaves identically and prevents intracellular Na⁺ and Ca⁺⁺ loading during PMI, ODSH applied externally to the myocyte membrane by addition to the medium was also behaving pharmacologically as a Na⁺ channel inhibitor to reduce late inward sodium current I_{Na}. Other research studying ranolazine has found that a major component of Na⁺ loading that occurs in this model during P-MI is inhibited by ranolazine (Zhang et al, 2008), and thus appears to be mediated by Na⁺ influx via the late Na⁺ current, I_{Na,L} (Antzelevitch et al, 2004). This increased Na⁺ loading causes increased Ca²⁺ loading via NCX.

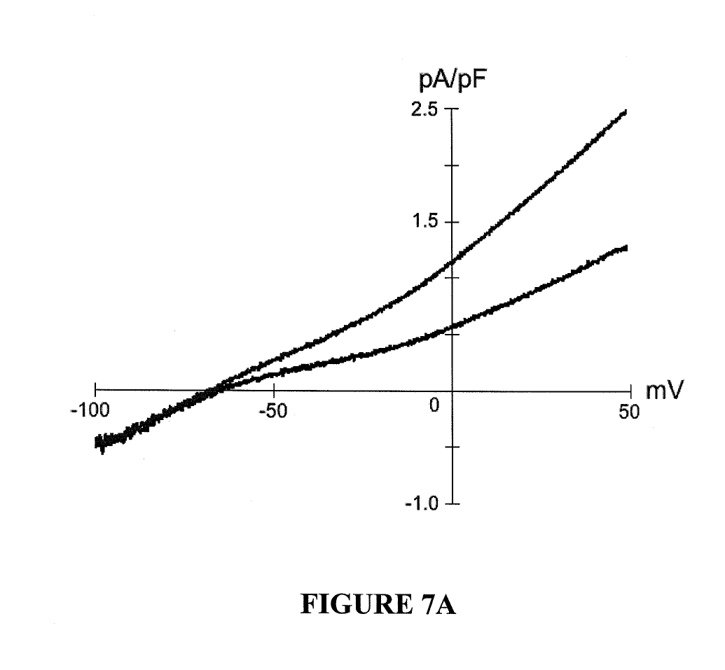

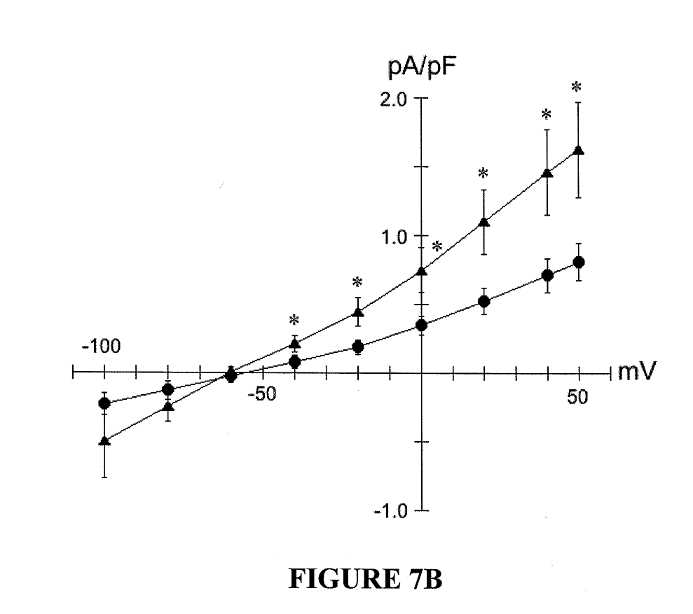

The direct effects of ODSH on NCX were further measured by means of voltage clamp studies in intact isolated rabbit ventricular myocytes. The results are shown in FIGS. 7A and 7B. Rather than inhibiting NCX, ODSH caused a significant stimulation of exchange. This finding was initially surprising but is consistent with a previous report that heparin and heparan sulfate disaccharides can stimulate Ca²⁺ extrusion by NCX in smooth muscle cell lines (Shinjo et al, 2002). These results also indicated that ODSH was not reducing Ca²⁺ loading during P-MT by direct inhibition of the exchanger. We therefore studied the effects of ODSH on Na⁺ loading.

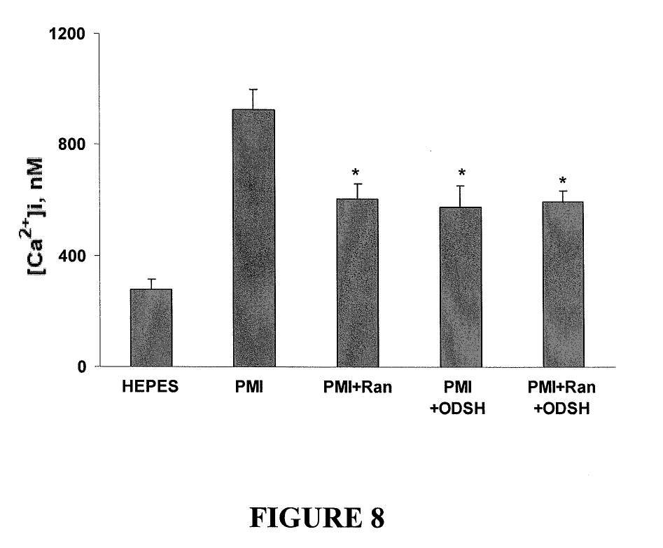

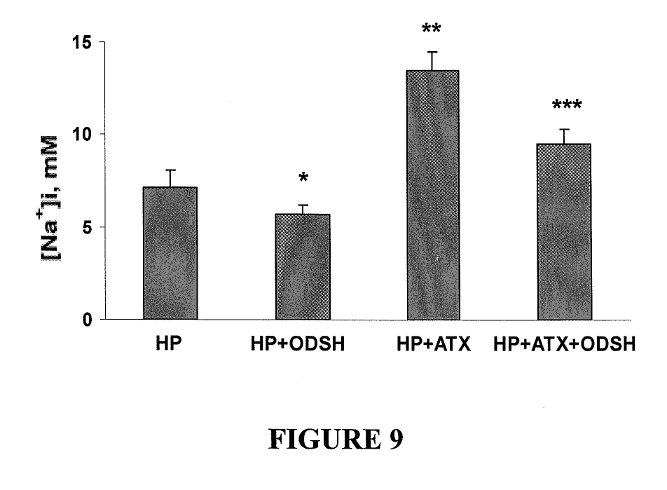

To determine if ODSH could be altering Na⁺ loading via a similar inhibitory effect on I_{Na,L}, the effects of ODSH on [Ca²⁺]ᵢ during P-MI in the presence of ranolazine were examined. The results are shown in FIG. 8. ODSH and ranolazine (Ran) reduced [Ca²⁺]ᵢ to a similar degree, and in the presence of ranolazine, ODSH had no additional effect on Ca²⁺ loading. These observations provided strong indirect evidence that ODSH was decreasing Na⁺ (and Ca²⁺) loading via an inhibition of I_{Na,L}. To provide more support for this idea, the effects of a selective activator of I_{Na,L}, sea anemone toxin II (ATX) (Schriebmakyer et al, 1987), on [Na⁺]ᵢ in paced myocytes in the absence of metabolic inhibition were examined. The results are shown in FIG. 9. Exposure to 10 nM ATX caused a substantial increase in [Na⁺]ᵢ in paced myocytes that was almost completely inhibited by ODSH 100 ug/ml. ODSH also reduced [Na⁺]ᵢ in myocytes in the absence of ATX but to a much smaller extent. ODSH 100 μg/ml had no effect of fluorescence intensity of fluoresceine-labeled microspheres, indicating there was no quenching of Na Green (or Fluo-3) fluorescence by ODSH.

The experiments outlined above indicate that ODSH is a potent treatment to prevent injurious intracellular Na⁺ and secondarily Ca⁺⁺ accumulation occurring as the consequence of ischemia. The studies outlined indicate that ODSH used in concentrations of about 100 μg/mL would provide effective treatment for ischemia in a wide variety of organs, tissues and whole organisms.

### Example 2

### Reduction in Ischemic Cardiac Necrosis by 2-O, 3-O Desulfated Heparin in a Porcine Closed Chest Model

To study the utility of 2-O, 3-O desulfated heparin (ODSH) in reducing ischemic tissue injury from injurious Ca⁺⁺ overload, a closed chest porcine model of cardiac ischemia was used. The study was designed to determine if previous findings indicating the protective effect of ODSH in reducing myocardial infarction reperfusion injury in open chest dogs were reproducible in an animal model of ischemia/reperfusion injury more relevant to humans. The results indicate that 2-O, 3-O desulfated heparin, when given just before relief of ischemia, reduces myocardial necrosis and the size of myocardial infarction in a pig model of this disease. The protective effects observed with pharmacological preconditioning with ODSH have been attributed to the anti-inflammatory activity of heparins, since ODSH impairs neutrophil rolling through inhibition of P- and L-selectins, and also significantly reduces neutrophil influx into ischemic reperfused myocardium. Yorkshire-cross pigs (Palmetto Research Swine, Reevesville, S.C.) of either sex, weighing 25-35 kg, were used for the experiment was employed. Animals were premedicated with an intramuscular injection of ketamine (30 mg/kg), acepromazine (1.1 mg/kg), and atropine (0.05 mg/kg). Pigs were induced with an IV injection of thiopental (10 mg/kg) and maintained with continuous inhalation of isoflurane (1-1.5%). Aspirin (81 mg) was administered by IV prior to the experiment.

Arterial access was achieved via bilateral femoral artery cut-downs for the insertion of 8F sheaths. Central venous and carotid access was achieved via a neck incision to expose the external jugular vein and common carotid artery. Animals were then anticoagulated with 50 U/kg of unfractionated heparin to maintain an activated clotting time (ACT) between 250-350 seconds, prior to ischemia. A 7-8 Fr pigtail catheter was placed in the left ventricular cavity to measure pressure and for injection of 15 μneutron-activated microspheres. Angiography was performed to define coronary anatomy and measure the diameter of the left anterior descending coronary artery (LAD) at the point of intended balloon occlusion. A coronary sinus catheter was placed via the external jugular vein under fluoroscopic guidance for coronary venous sampling. Baseline cardiodynamic and hemodynamic data were measured using a solid state transducer-tipped catheter in the left ventricle (Millar Instruments, Houston, Tex.) to measure left ventricular pressure, and a fluid-filled transducer connected to the side port of the femoral artery sheath to measure peripheral arterial pressure. Approximately 3-4 million neutron-activated microspheres (BioPhysics Assay Laboratory, Inc, Worcester, Ma) (15 μm) were delivered through a pig-tail catheter into the left ventricle over a 30 second period, to quantify regional myocardial blood flow (RMBF). Simultaneously with injection, a reference sample was withdrawn at a rate of 7 cc/min from the femoral artery sheath, for 90 seconds during and after injection of microspheres. A contrast ventriculogram (60° right anterior oblique) was obtained to assess global and regional myocardial function at baseline. An angioplasty balloon catheter sized to exceed ambient diameter by 1 mm (range chosen was 3.0-4.0 mm) was then inserted into the proximal LAD, after the first diagonal branch. Prior to ischemia, amiodarone (8 mg/kg) was administered to reduce the incidence of ischemia-related ventricular arrhythmias so that fatal cardiac rhythm disturbances, common in cardiac ischemia, did not prevent completion of the remainder of the experiment. In addition, 2% lidocaine (4-8 ml total) was administered during ischemia, as needed, to attenuate ventricular arrhythmias. Balloon occlusion time was 75 minutes with coronary occlusion confirmed by contrast angiography, ST segment changes on electrocardiography, and quantitatively confirmed by microspheres delivered at the end of ischemia, as described above. Episodes of ventricular fibrillation were immediately treated with electrical cardioversion delivered at 200 Joules. During the ischemic period, animals were randomly assigned to receive either saline vehicle or 2-O, 3-O desulfated heparin (ODSH) at a dose of 5 mg/kg, 15 mg/kg, or 45 mg/kg as an IV bolus at 2 minutes prior to deflation of the balloon (pharmacological postconditioning), and repeated at 90 minutes of reperfusion. Following deflation of the angioplasty balloon, animals underwent 3 hours of further reperfusion and observation. Microspheres were again injected to measure myocardial blood flow at 15 minutes of reperfusion and again at 180 minutes of reperfusion. At the end of 180 minutes of reperfusion, animals were euthanized with an IV injection of pentobarbital sodium (100 mg/kg) and the heart was excised to quantify the area at risk, infarct size, regional myocardial blood flow, and myeloperoxidase activity. Hemodynamic data (left ventricular and arterial blood pressure) and derived variables were recorded continuously using 10× and Datanalyst software (EMKA Technologies, Falls Church, Va.).

After harvesting the heart, the LAD was ligated with a 2-0 silk suture placed at the site of balloon inflation, and diluted (5%) Unisperse blue dye was injected into the aortic root to stain the non-ischemic region blue and thereby outline the area at risk (AAR). The left ventricle was then cut into 5-6 transverse slices and the AAR was separated from the non-ischemic zone and incubated in a 1% buffered solution of triphenyltetrazolium chloride (TTC) at 37° C. to differentiate the area of necrosis from the non-necrotic AAR. The AAR, as a percent of the left ventricular mass (AAR/LV), and the area of necrosis (NEC), as a percent of the AAR (NEC/AAR), were calculated by tissue weight as reported previously (Thourani et al., Amer. J. Physiol. Heart Circ. Physiol. 48: H2084-2093 (2000)).

After determining infarct size, tissue samples from the non-ischemic and area at risk zones were saved for analysis of myeloperoxidase (MPO) activity, an enzyme used as a marker of neutrophil accumulation. The samples were frozen and stored at −70° C. until assayed. The samples were homogenized in hexadecyltrimethyl ammonium bromide and dissolved in potassium phosphate. After centrifugation, supernatants were collected and mixed with O-dianisidine dihydrochloride and hydrogen peroxide in phosphate buffer. The activity of MPO was measured spectrophotometrically at 460 nm absorbance (SPECTRAmax, Molecular Devices, Sunnyvale, Calif.) and expressed as Δabs/min/g tissue (Thourani et al., Amer. J. Physiol. Heart Circ. Physiol. 48: H2084-2093 (2000)).

Regional myocardial blood flow in the subepicardial and subendocardial regions of the AAR and non-ischemic left ventricular free wall was determined by neutron-activated microspheres at baseline, ischemia, and at 15 minutes and 3 hours of reperfusion, using the reference sampling method as previously described (Zhao Z-Q, et al. Am. J. Physiol. Heart Circ. Physiol. 285:H579-H588, 2003). Samples were desiccated according to instructions from BioPal Laboratories, and sent for activation and analysis. Results are expressed as ml/min/g tissue determined from the equation:

Flow_{T}=[(R_{T}×Flow_{Ref})/R_{Ref}]/Weight_{T}, where T=tissue, R=radioactivity which is≈the number of microspheres, and Ref=reference sample.

Blood anticoagulation was measured by following the ACT, determined using the Hemochron whole blood coagulating system (Hemochron, Edison, N.J.) and measured 10 minutes after delivery of 50 U/kg of unfractionated heparin after the insertion of arterial sheaths. Subsequent ACTs were obtained 10 minutes after delivery of either saline vehicle or ODS and at 3 hours of reperfusion.

Left ventricular contrast angiography was performed at baseline, ischemia, and 180 minutes after relief of ischemia. Contrast (Hypaque, approximately 50 mL) was rapidly injected via a pigtail catheter using a power injector. Left ventricular ejection fraction (LVEF) and regional function of the antero-lateral wall was calculated using the area-length method, which outlined the ventricle at the end of systole and diastole. LVEF and regional function were analyzed independently by 2 blinded observers.

Data are expressed as the mean±standard error. A one-way analysis of variance (ANOVA) followed by Student-Newman-Keuls post hoc test was used to analyze for group differences in single point data such as infarct size, myocardial edema, creatine kinase, and MPO. Repeated measures data from hemodynamics, ventriculography, and regional blood flow were analyzed by repeated measures of analysis of variance followed by post-hoc analysis with Student-Newman-Keuls for multiple comparisons. A P level of <0.05 was assigned significance.

Forty-three pigs were initially entered into the study. A priori exclusion criteria were established to exclude cases in which the area at risk (AAR/LV) was <20% or >50%. Based on these exclusion criteria, 3 animals were excluded for AAR/LV<20% and 1 for AAR/LV>50%. In addition, 1 animal was excluded because the distal microcirculation failed to demonstrate blood flow following balloon deflation (microspheres), and 3 were excluded because of technical complications (perivascular hematoma, cardiac tamponade, and intractable reperfusion arrhythmias). Six animals died during ischemia, from intractable ventricular fibrillation. Data from 29 pigs are included in the final analysis: 8 vehicle (Control), 6 ODSH 5 mg/kg (ODS 5), 8 ODSH 15 mg/kg (ODS 15), and 7 ODSH 45 mg/kg (ODS 45).

Regional myocardial blood flow was equivalent in the non-ischemic myocardium at baseline in all groups studied. Myocardial blood flow in the non-ischemic left ventricular myocardium remained unchanged during ischemia and following ischemia. LAD occlusion reduced subendocardial blood flow in the area at risk by >99% for all groups, with no group differences. There were no significant group differences in the area at risk of regional blood flow in either the subepicardial or subendocardial regions at baseline, end ischemia, or at 15, 60, or 180 minutes of reperfusion.

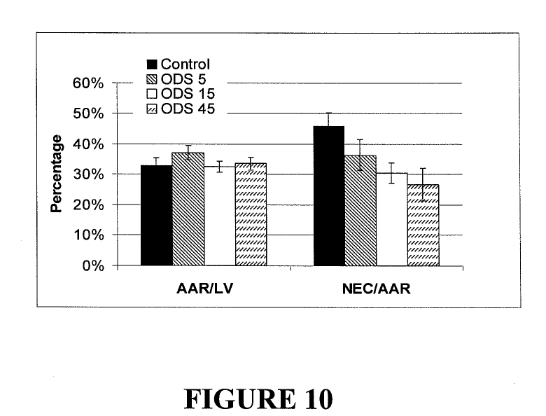

The individual data on AAR and AN are presented in FIG. 10. The average data for AAR/LV was similar among all groups (FIG. 10, left panel). No significant reduction in infarct size was observed in the 5 mg/kg ODSH group, compared to control. However, there was a significant infarct size reduction (NEC/AAR) with both 15 mg/kg and 45 mg/kg ODSH (relative to control) with no difference between groups receiving 15 or 45 mg/kg ODSH (See FIG. 10, right panel). Since collateral blood flow during ischemia was comparable in all groups, the significant reduction in infarct size in the two treatment groups was not due to greater values in collateral blood flow during coronary occlusion. There may be no effect of the ODSH heparin on collateral blood flow since the compound was not administered until 5 minutes before reperfusion.

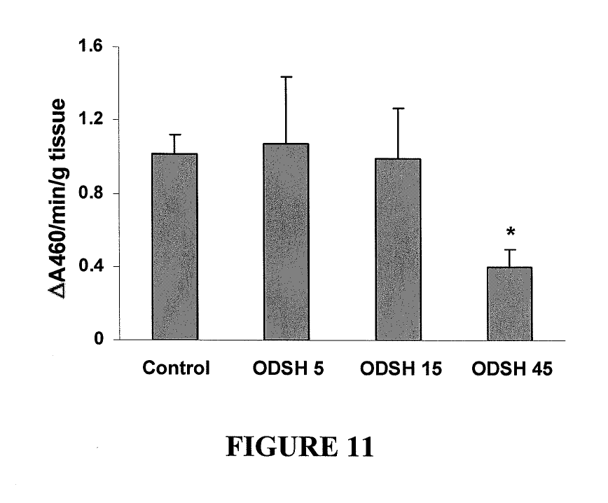

It has been previously demonstrated that ODSH reduces canine myocardial reperfusion injury accompanied by a significant reduction in neutrophilic infiltration into ischemic reperfused myocardium (Thourani et al, Amer. J. Physiol. Heart Circ. Physiol. 48: H2084-H2093 (2000)). It was therefore expected that similar results in ischemic-reperfused pigs would be seen. Surprisingly, MPO activity in ischemic-reperfused myocardium was significantly reduced compared to Controls only in pigs treated with 45 mg/kg ODSH, and not in pigs receiving 15 mg/kg drug (FIG. 9). In non-ischemic myocardium, MPO activity was comparable among all groups (data not shown). These results confirm previous findings that ODSH reduced infarct size in open chest dogs when given 5 min prior to reperfusion. However, reduction in neutrophil influx does not appear to be the only mechanism of protection, since significant infarct size reduction was observed with 15 mg/kg ODSH (FIG. 10) despite no decrease in myocardial MPO (FIG. 11).

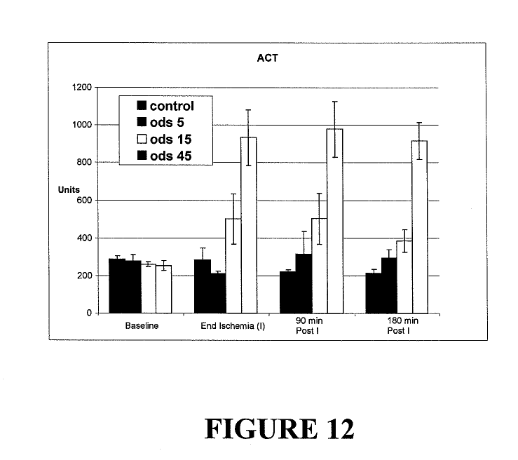

ACT data are shown in FIG. 12. There were no significant group differences in ACT at baseline, with mean values between 250-350 seconds following administration of unfractionated heparin to prevent clot formation on angioplasty catheters. Similarly there were no significant differences in ACT at end ischemia, or 90 minutes and 180 minutes following the end of ischemia among control, ODS 5, and ODS 15 groups. However, ACT was significantly higher in ODS 45 compared to the other groups at end ischemia, and 90 or 180 minutes after the end of ischemia. End ischemia values represent ACT analysis performed 10 minutes after delivery of either saline vehicle or ODS. The elevation above control in ACT in the 15 mg/kg dose group (approximately 200 seconds) is the degree of elevation required to effect appropriate anti-coagulation in the cardiac catheterization laboratory or in the early hours after myocardial infarction in humans.

TABLE 9
Global Ejection Fraction Determined by Contrast Ventriculography

| | | | 180 Minutes after |
|---|---|---|---|
| | Baseline | Ischemia | End of Ischemia |
| Control | 52 ± 2% | 25 ± 2% | 34 ± 3% |
| ODS 15 mg/kg | 61 ± 2% | 30 ± 3% | 42 ± 5% |
| ODS 15 mg/kg | 57 ± 4% | 23 ± 3% | 42 ± 4% |
| ODS 45 mg/kg | 60 ± 2% | 30 ± 3% | 38 ± 6% |

Ejection fraction, determined by contrast angiography, was similar at baseline among all groups (Table 9 above). Moreover, ejection fraction was comparably reduced by ˜50% at the end of ischemia for all groups compared to their respective baseline. Ejection fraction at 3 hours after the end of ischemia remained lower than baseline in all groups but tended to be higher in ODS-treated animals. There were no significant differences in left ventricular systolic or end-diastolic pressure, heart rate, or mean arterial pressure at any of the time points.

### Example 3

2-O Desulfated Heparin does not Activate Platelets in the Presence of Heparin-Induced Thrombocytopenia Antibody

To be used safely in the treatment of prevention or treatment of ischemic-induced Ca⁺⁺ overload, a heparin derivative would have to be free of the dangerous side effect of inducing heparin-induced thrombocytopenia type II, referred to as HIT. It was determined whether 2-O desulfated heparin was free from HIT activation properties usually manifested by unfractionated heparin (UFH). The potential of 2-O desulfated heparin (ODSH) to interact with HIT antibody and active platelets was studied using donor platelets and serum from three different patients clinically diagnosed with HIT, by manifesting thrombocytopenia related to heparin exposure, correction of thrombocytopenia with removal of heparin, and a positive platelet activation test, with or without thrombosis. Two techniques were employed to measure platelet activation in response to heparin or 2-O desulfated heparin in the presence of HIT-reactive serum.

The first technique was the serotonin release assay (SRA), considered the gold standard laboratory test for HIT, and performed as described by Sheridan (Sheridan D, et al. Blood 67:27-30, 1986). Washed platelets were loaded with ¹⁴C serotonin (¹⁴C-hydroxy-tryptamine-creatine sulfate, Amersham), and then incubated with various concentrations of test heparin or heparin analog in the presence of serum from known HIT-positive patients as a source of antibody. Activation was assessed as ¹⁴C serotonin release from platelets during activation, with ¹⁴C serotonin quantitated using a liquid scintillation counter. Formation of the heparin-PF4-HIT antibody complex resulted in platelet activation and isotope release into the buffer medium. Activated platelets are defined as percent isotope release of ≧20%.

Specifically, using a two-syringe technique, whole blood was drawn from a volunteer donor into sodium citrate (0.109M) at a ratio of 1 part anticoagulant to 9 parts whole blood. The initial 3 ml (milliliters) of whole blood in the first syringe was discarded. The anticoagulated blood was centrifuged (80×g (gravity), 15 min, room temperature) to obtain platelet rich plasma (PRP). The PRP was labeled with 0.1 μCuries ¹⁴Carbon-serotonin/ml (45 min, 37° C.), then washed and resuspended in albumin-free Tyrode's solution to a count of 300,000 platelets/μL (microliter). HIT serum (20 μl) was incubated (1 hour@room temperature) with 70 μl of the platelet suspension, and 5 μl of 2-O desulfated heparin (0, 0.78, 1.56, 3.13, 6.25, 12.5, 25, 50 and 100 μg (micrograms)/mL final concentrations). For system controls, 10 μL unfractionated heparin (UFH; either 0.1 or 0.5 U/ml final concentrations, corresponding to the concentrations in plasma found in patients on anti-thrombotic or fully anticoagulant doses, respectively) was substituted for the 2-O desulfated heparin in the assay. EDTA was added to stop the reaction, and the mixture was centrifuged to pellet the platelets. ¹⁴C-serotonin released into the supernatant was measured on a scintillation counter. Maximal release was measured following platelet lysis with 10% Triton X-100. (Sigma Chemicals, St. Louis, Mo.). The test was positive if the release was ≧20% serotonin with 0.1 and 0.5 U/ml UFH (no added 2-O desulfated heparin) and <20% serotonin with 100 U/ml UFH. The test was for cross-reactivity of the HIT antibodies with the 2-O desulfated heparin if ≧20% serotonin release occurred.

The second technique employed was flow cytometric platelet analysis. In this functional test, platelets in whole blood are activated by heparin or heparin analog in the presence of heparin antibody in serum from a patient clinically diagnosed with HIT. Using flow cytometry, platelet activation was determined in two manners: by the formation of platelet microparticles and by the increase of platelet surface bound P-selectin. Normally, platelets in their unactivated state do not express CD62 on their surface, and platelet microparticles are barely detectable. A positive response is defined as any response significantly greater than the response of the saline control.

Specifically, whole blood drawn by careful double-syringe technique was anticoagulated with hirudin (10 μg/ml final concentration). An aliquot of whole blood (50 μL) was immediately fixed in 1 ml 1% paraformaldehyde (gating control). HIT serum (160 μL) and 2-O desulfated heparin (50 μl; 0, 0.78, 1.56, 3.13, 6.25, 12.5, 25, 50 and 100 μg/mL final concentrations) were added to the whole blood (290 μl) and incubated (37° C., 15 minutes, with stirring at 600 rpm). Aliquots (50 μL) were removed and fixed in 1 mL paraformaldehyde (30 minutes, 4° C.). The samples were centrifuged (350 g, 10 minutes) and the supernatant paraformaldehyde removed. The cells were resuspended in calcium-free Tyrode's solution (500 μL, pH 7.4±0.1). 150 μL cell suspension was added to 6.5 μL fluorescein isothiocyanate (FITC) labeled anti-CD61 antibody (Becton-Dickinson; San Jose, Calif.; specific for GPIIIa on all platelets). Samples were incubated (30 minutes, room temperature) in the dark. All antibodies were titrated against cells expressing their specific antigen prior to experimentation to assess the saturating concentration. Samples were analyzed on an EPICS XL flow cytometer (Beckman-Couter; Hialeah, Fla.) for forward angle (FALS) and side angle light scatter, and for FITC and PE (phycoerythrin) fluorescence. Prior to running samples each day, a size calibration was made by running fluorescent-labeled beads of known size (Flow-Check; Coulter) and adjusting the gain so that 1.0 μm beads fall at the beginning of the second decade of a 4-decade log FALS light scatter scale. A threshold discriminator set on the FITC signal was used to exclude events not labeled with anti-CD61 antibody (non-platelets).

Using the gating control sample, amorphous regions were drawn to include single platelets and platelet microparticles. Platelet microparticles were distinguished from platelets on the basis of their characteristic flow cytometric profile of cell size (FALS) and FITC fluorescence (CD61 platelet marker). Platelet micro-particles were defined as CD61-positive events that were smaller than the single, nonaggregated platelet population (<˜1 μm). 20,000 total CD61-positive events (platelets) were collected for each sample. Data was reported as a percentage of the total number of CD61-positive events analyzed. In testing for cross-reactivity with a heparin-dependent HIT antibody, the UFH controls (no 2-O desulfated heparin) should show a positive response (increased percentage of CD61 positive events in the platelet microparticle region at 0.1 and 0.5 U/mL UFH, but not at 100 U/mL UFH). The test was positive for cross-reactivity of the HIT antibodies with the 2-O desulfated heparin if an increase in platelet microparticle formation occurred.

The quantitation of P-selectin expression induced on the surface of platelets by HIT-related platelet activation was determined as follows. To quantitate platelet surface expression of P-selection, platelet-rich plasma was collected and platelets were labeled as described above, but additionally labeled with 6.5 μl of phycoerythrin (PE) labeled antibody (Becton-Dickinson; specific for P-selectin expressed on activated platelets). The gating control sample was used to establish the regions of single platelets and platelet microparticles based on FALS and CD61-FITC fluorescence. A histogram of PE fluoresce (P-selectin expression) was gated to exclude platelet aggregates. A marker encompassing the entire peak was set in order to determine the median P-selectin fluorescence. Results were reported in mean fluorescence intensity units (MFI) of CD62 in the non-aggregated platelet population. In testing for cross-reactivity with a heparin-dependent HIT antibody, the UFH controls should show a positive response (increased median P-selectin fluorescence) at 0.1 and 0.5 U/mL UFH but not at 100 U/mL UFH. The test was positive for cross-reactivity of the HIT antibodies with the 2-0 desulfated heparin if an increase in platelet P-selectin expression occurred.

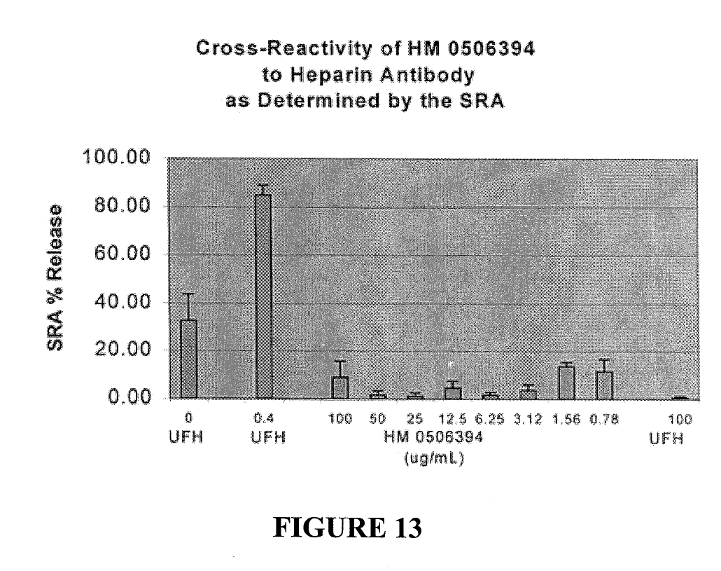

FIG. 13 shows that unfractionated heparin (UFH) at the usual therapeutic anticoagulant concentration of 0.4 μg/ml elicited release of >80% of total radio labeled serotonin in this system. In contrast, the 2-O desulfated heparin (manufactured lot HM0506394), studied in a range of concentrations from 0.78 to 100 μg/mL, failed to elicit substantial ¹⁴C serotonin release, indicating that this 2-O desulfated heparin does not interact with a pre-formed HIT antibody causing platelet activation. The interaction of regular heparin with the HIT antibody caused platelet activation. When ODSH was added with heparin to the HIT antibody, the ODSH prevented heparin from causing platelet activation.

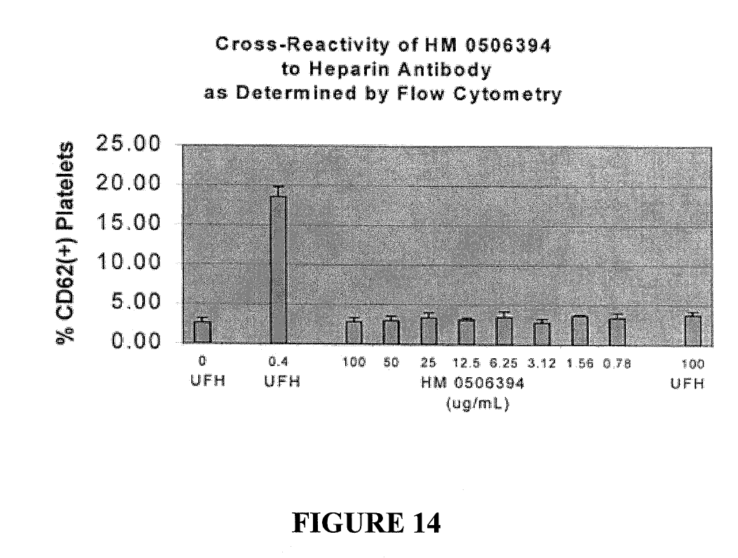

FIG. 14 shows that when unfractionated heparin (UFH) at the usual therapeutic anticoagulant concentration of 0.4 μg/mL was incubated with platelets and HIT-antibody positive serum, there was prominent CD62 expression on the surface of approximately 20% of the platelets. Saline control incubations were characterized by low expression of CD62 (<2% of platelets). In contrast, 2-O desulfated heparin (manufactured lot HM0506394), studied at 0.78 to 100 μg/mL, did not increase CD62 expression levels above that observed in the saline control incubations. Furthermore, while 0.4 μg/mL unfractionated heparin produced substantial platelet microparticle formation, 2-O desulfated heparin at 0.78 to 100 μg/mL stimulated no level of platelet microparticle formation above that of the saline control incubations (<5% activity).

These results provide the surprising and beneficial finding that 2-O desulfated heparin can be used to treat or prevent ischemic Na⁺ and secondarily Ca⁺⁺ overload in patients suffering the same without inducing the dangerous side effect of thrombotic events from heparin-induced thrombocytopenia. Because no other heparin derivative is known to be free of HIT activating activity, 2-O desulfated heparin is particularly useful as a clinical heparin therapy for treating ischemia because of its clinical safety.

### Example 4

### Safe, Intravenous Bolus Administration of 2-O, 3-O Desulfated Heparin to Humans

A study was performed in 38 volunteer human subjects to assess the safety of escalating bolus doses of 2-O, 3-O desulfated heparin (ODSH). The study was a Phase I, randomized, double-blind, dose-escalation study with a single-day treatment period. Subjects were between the ages of 18 and 45, were not pregnant, and were normal in body weight. They all had normal coagulation function and hemoglobin values at baseline.

Doses within treatment groups were not escalated, and subjects received a single intravenous dose of ODSH over 15 minutes of either active drug or placebo. Two subjects also received an injection of fully anticoagulated unfractionated heparin for comparison. ODSH dose groups were run in a series, and safety and tolerance data were evaluated prior to the start of the next dose level (4, 8 12, 16 and 20 mg/kg bolus intravenous doses). Twenty eight (28) subjects randomly received ODSH and 9 subjects were randomized to receive placebo, with an additional two subjects receiving commercially available unfractionated heparin. Dosing was performed according to the schedule shown in Table 10.

TABLE 10

| | | M:F | Active/Placebo | Active | Active Agent | Dose |
|---|---|---|---|---|---|---|
| Group | n | Ratio | Ratio | Agent | Dose (mg/kg) | (U/kg) |
| 1 | 8 | 4:4 | 3:1 | ODSH | 4 or 0 | na |
| | | | (within gender) | | | |
| 2 | 8 | 8:0 | 3:1 | ODSH | 8 or 0 | na |
| 3 | 8 | 8:0 | 3:1 | ODSH | 12 or 0 | na |
| 4 | 8 | 8:0 | 3:1 | ODSH | 16 or 0 | na |
| 5 | 5 | 5:0 | 4:1 | ODSH | 20 or 0 | na |
| 6 | 2 | 2:0 | 2:0 | Unfrac- | 0.571 | 80 |
| | | | | tionated | | |
| | | | | heparin | | |

1 mg heparin = 140 units

For each bolus dose, ODSH as a 50 mg/mL formulation was diluted with normal saline and a total volume of 50 ml was infused over 15 minutes containing the calculated amount of ODSH the subject was to receive. Placebo consisted of 50 mL of normal saline infused over 15 minutes. For subjects receiving heparin, 5,000 units (approximately 0.5 mg/kg) of heparin was diluted into 50 ml of normal saline and infused over 15 minutes.

Immediately before infusion and beginning 7 minutes after the start of each infusion, blood was drawn at periodic times for 24 hours to monitor the effect of infusion on the following laboratory studies: activated partial thromboplastin time (aPTT); prothrombin time (PT); activated clotting time (ACT); and ODSH plasma level. Serum chemistries and a complete blood count were checked immediately before infusion and at eight (8) and twenty-four (24) hours later. Using values for aPTT and ODSH levels, pharmacokinetic parameters were calculated by noncompartmental methods using a commercial software program (PhAST 2.3-001). The following pharmacokinetic parameters were calculated:
a) Maximum measured plasma concentration (Cₘₐₓ);
b) First-order terminal elimination rate constant (Kel), calculated from a semi-log plot of the serum concentration versus time curve; this parameter was calculated by linear least-square regression analysis using the maximum number of points in the terminal log-linear phase (e.g., 3 or more non-zero serum concentrations);
c) Time of the maximum measured drug plasma concentration (tₘₐₓ);
d) The area under the plasma concentration versus time curve from time 0 to the last observation (AUC 0-t), calculated by the linear trapezoidal method;
e) The area under the plasma concentration versus time curve from time 0 to infinity (AUCinf), which was calculated as the sum of AUC 0-t plus the ratio of the last measurable serum concentration to the elimination rate constant;
f) First-order terminal elimination (t_{1/2}), calculated as 0.693/Kel;
g) Total body clearance (CL), calculated as Dose/AUCinf, and
h) Total volume of distribution (Vdss), calculated as MRT×CL.

No serious adverse events were noted and none of the subjects were discontinued from the study due to an adverse event. No treatment- or dose-related trends were noted in the serum chemistry, hematological, urinalysis, or physical exam findings. Specifically, bolus ODSH did not increase blood glucose, nor did it elevate blood pressure. Mean ACT value at 15 minutes for the two heparin treated patients receiving about 0.5 mg/kg heparin was 333 seconds; however, the mean ACT value for subjects receiving 20 mg/kg ODSH was only 207 seconds (a difference of over 100 seconds, even though the drug dose was 40-fold higher). Thus, ODSH is substantially less anticoagulating than unfractionated heparin.

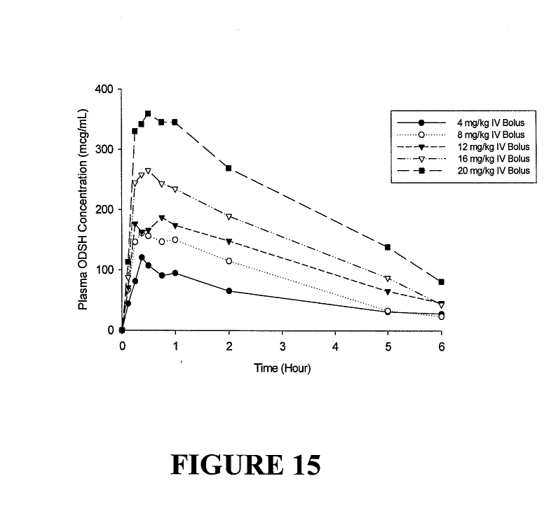

The mean plasma concentrations of ODSH for the dose levels studied are presented in FIG. 15. ODSH plasma concentrations peaked shortly after the end of infusion and then declined in an exponential manner. Descriptive statistics of the pharmacokinetic parameters of ODSH in this study are summarized below in Table 11. Mean clearance values of ODSH were consistent throughout the dose range studied (values ranged from 10.3 to 15.4 mL/h/kg), indicating a dose proportional increase in pharmacokinetic parameters over the dose range studied. Mean elimination half-life values of ODSH from 4 to 20 mg/kg were short, with mean values ranging from 1.93 to 2.72 hours. Median tₘₐₓ values of ODSH were observed shortly after the end of the infusion period. Tₘₐₓ values were comparable over the dose range of 4 to 20 mg/kg, with values ranging from 0.37 to 0.88 hours.

TABLE 11

| | ODSH Dose Levels | | | | |
|---|---|---|---|---|---|
| | Group 1 | Group 1 | Group 3 | Group 4 | Group 5 |
| Pharmacokinetic | 4 mg/kg | 8 mg/kg | 12 mg/kg | 16 mg/kg | 20 mg/kg |
| Parameters | (n = 6) | (n = 6) | (n = 6) | (n = 6) | (n = 4) |

Geometric Mean CV %

| | | | | | | | | | | |
|---|---|---|---|---|---|---|---|---|---|---|
| AUC 0-t (μg h/mL) | 307.2 | (52.9%) | 461.9 | (46.9%) | 619.1 | (62.3%) | 886.9 | (21.2%) | 1322.1 | (7.8%) |
| AUCinf (μg h/mL) | 415.2 | (44.2%)* | 629.2 | (18.2%)** | 1086.5 | (19.7%)* | 1075.8 | (29.4%) | 1638.7 | (6.5%) |
| Cₘₐₓ (μg/mL) | 130.76 | (34.1%) | 163.74 | (19.7%) | 179.28 | (66.8%) | 285.38 | (13.5%) | 366.73 | (9.7%) |

Arithmetic Mean +/− SD

| | | | | | |
|---|---|---|---|---|---|
| t_{1/2} (h) | 2.585 ± 1.1225* | 1.933 ± 0.4537** | 2.724 ± 0.6667* | 2.261 ± 0.8548 | 2.637 ± 0.4765 |
| CL (mL/h/kg) | 10.254 ± 3.8984* | 12.882 ± 2.3521** | 11.202 ± 2.1722* | 15.364 ± 4.0502 | 12.526 ± 0.2090 |
| Vdss (mL/kg) | 34.95 ± 11.679* | 35.13 ± 6.580** | 42.12 ± 3.170* | 47.25 ± 7.944 | 45.56 ± 8.256 |
| MRT (h) | 3.780 ± 1.6710* | 2.775 ± 0.6087** | 3.894 ± 0.9516* | 3.287 ± 1.0560 | 3.639 ± 0.6670 |

Median (Min-Max)

| | | | | | | | | | | |
|---|---|---|---|---|---|---|---|---|---|---|
| tₘₐₓ (h) | 0.47 | (0.25-1.00) | 0.37 | (0.25-0.62) | 0.88 | (0.25-2.00) | 0.50 | (0.37-0.75) | 0.50 | (0.37-1.00) |

*For these parameters n = 4;
**For these parameters n = 5

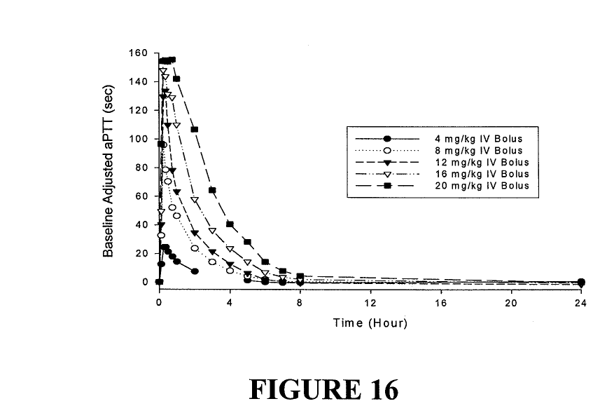

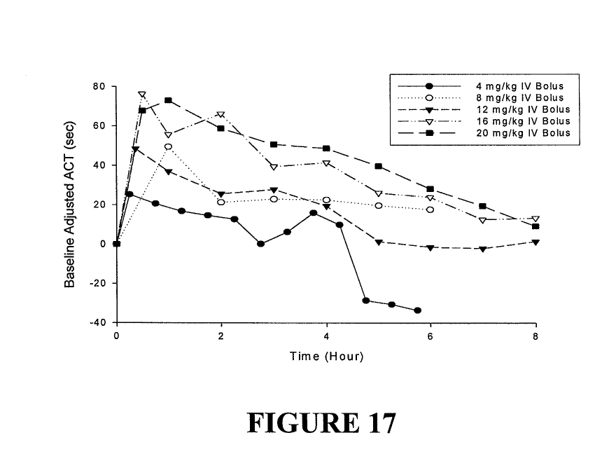

The change from baseline in aPTT is shown in FIG. 16. ODSH produced a rapid increase in aPTT over the infusion period in a dose-dependent fashion. The change from baseline in ACT is shown in FIG. 17. Therapeutic increases in the ACT appropriate for anticoagulation treatment of patients undergoing cardiac catheterization were observed with ODSH bolus doses of 12-20 mg/kg. PT also increased in a dose-dependent manner.

Platelet counts for ODSH- and placebo-treated patients in all dose groups are shown below in Table 12, wherein values are provided as thousands/μL blood (mean±SD). ODSH did not produce the >50% fall in platelet counts characteristic of heparin-induced thrombocytopenia (HIT), indicating that this heparin analog (ODSH) is safe from producing HIT during use at clinical doses in humans.

TABLE 12

| | |
|---|---|
| Before Bolus | 24 h After |
| Dose | Bolus Dose |

| Dose | ODSH | Placebo | ODSH | Placebo |
|---|---|---|---|---|
| 4 mg/kg | 267 ± 72 | 287 ± 84 | 207 ± 70 | 267 ± 73 |
| 8 mg/kg | 248 ± 39 | 258 ± 30 | 236 ± 34 | 257 ± 8 |
| 12 mg/kg | 236 ± 63 | 293 ± 82 | 221 ± 52 | 309 ± 91 |
| 16 mg/kg | 260 ± 37 | 242 ± 47 | 252 ± 37 | 242 ± 71 |
| 20 mg/kg | 288 ± 27 | 278 | 278 ± 34 | 274 |

These data demonstrate that ODSH is safe when administered at large bolus doses, producing ODSH plasma levels >300 μg/mL while at bolus doses of 12-20 mg/kg increasing the ACT to a therapeutic level of anticoagulation appropriate for subjects undergoing cardiac catheterization. Used in these bolus doses, ODSH also does not produce catastrophic thrombocytopenia characteristic of HIT. These data demonstrate safe doses of ODSH in humans that can be used to achieve blood levels of >100 μg/mL needed to inhibit injurious intracellular Na⁺ and secondarily Ca⁺⁺ overload from ischemia. At these doses subjects with ischemia, who are also often in need of anticoagulation, can be anticoagulated appropriate and therapeutic levels with ODSH doses presented.

### Example 5

### Safe Intravenous Bolus Administration and 12 Hour Infusion of 2-O, 3-O Desulfated Heparin to Normal Humans

A study was performed in twenty-four (24) healthy adult subjects to assess the effects of a bolus dose and 12 hour infusion of 2-O, 3-O desulfated heparin. The study was a Phase I, randomized, double-blind, dose escalation study with single-day treatment periods. Subjects were males between the ages of 18 and 45, and were normal in body weight. They all had normal coagulation function and hemoglobin values at baseline. Doses within treatment group were not escalated, and subjects received either active drug (ODSH) or placebo treatment. Eighteen (18) subjects were randomized to receive ODSH and six (6) subjects were randomized to receive placebo. Subjects received either ODSH or placebo as described below in Table 13.

TABLE 13

| | | | | Continuous Infusion |
|---|---|---|---|---|
| | | Active/Placebo | Bolus ODSH | ODSH |
| Group | n | Ratio | (mg/kg) | (mg/kg/12 hr) |
| 1 | 2 | 2:0 | 8 | 47.5 |
| 2 | 6 | 4:2 | 8 | 24 |
| 3 | 8 | 6:2 | 8 | 32 |
| 4 | 8 | 6:2 | 16 | 32 |

For each subject, ODSH as a 50 mg/ml formulation was diluted with normal saline and administered as a bolus infused over 15 minutes containing the calculated amount of ODSH the subject was to receive, followed by a constant infusion for 12 hours of ODSH diluted in saline. Placebo consisted of 50 mL of normal saline infused over 15 minutes, followed by normal saline infused for 12 hours. Immediately before infusion and after the start of each infusion, blood was drawn at periodic times (over a total 24 hour period) to monitor the effect of infusion on the following laboratory studies: activated partial thromboplastin time (aPTT); prothrombin time (PT); activated clotting time (ACT); and ODSH plasma level. Serum chemistries and a complete blood count were checked immediately before infusion and again periodically for up to twenty-four (24) hours later. Using values for aPTT and ODSH levels, pharmacokinetic parameters were calculated by noncompartmental methods using a commercial software program (PhAST 2.3-001). The following pharmacokinetic parameters were calculated (as described above): Cₘₐₓ; Kel; tₘₐₓ; AUC O-t; AUCinf, t_{1/2}; CL; and Vdss.

No serious adverse events were noted and none of the subjects were discontinued from the study due to an adverse event. No treatment- or dose-related trends were noted in the serum chemistry, hematological, urinalysis, or physical exam findings. Specifically, bolus ODSH did not increase blood glucose, nor did it elevate blood pressure. Mean plasma concentrations of ODSH for the bolus and infusion doses studied are presented in FIG. 16.

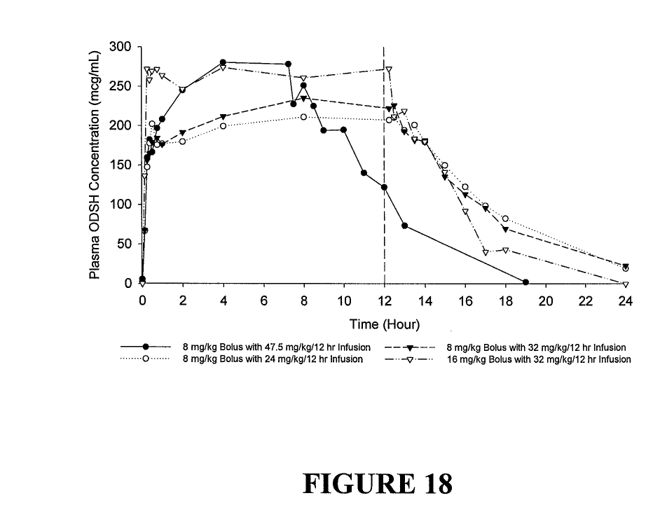

FIG. 18 summarizes plasma concentrations of ODSH for the treatment groups. All groups achieved sustained ODSH plasma concentrations of >100 μg/mL. ODSH plasma concentrations peaked shortly after the end of bolus infusion in all groups except those subjects who received 47.5 mg/kg over 12 hr (4 mg/kg/hr). These subjects had ODSH levels peak at about 275 μg/ml beginning approximately 4 hours after initiation of infusion. In this group, infusions were discontinued at 8 hours because of a rise in aPTT to sustained values greater than 120 seconds. After discontinuation of the infusion in this group, ODSH levels fell exponentially over the next 12 hours, as they did in the remaining three infusion dose groups. Descriptive statistics of the pharmacokinetic parameters of ODSH in this study for Groups 2 through 4 are summarized below in Table 14.

TABLE 14

| | ODSH Dose Levels | | |
|---|---|---|---|
| | Group 2 | Group 3 | Group 4 |
| | 8 mg/kg Bolus with | 8 mg/kg Bolus with | 16 mg/kg Bolus with |
| Pharmacokinetic | 24 mg/kg/12 hr infusion | 32 mg/kg/12 hr infusion | 32 mg/kg/12 hr infusion |
| Parameters | (n = 4) | (n = 6) | (n = 3) |

Geometric Mean CV %

| | | | | | | |
|---|---|---|---|---|---|---|
| AUC 0-t (μg h/mL) | 3,472.4 | 28.4% | 3,639.7 | 19.7% | 3,895.2 | 26.5% |
| AUCinf (μg h/mL) | 3,562.0 | 29.4% | 3,755.5 | 20.7% | 4,633.3 | N/C (n = 2) |
| Cₘₐₓ (μg/mL) | 216.79 | 19.4% | 246.39 | 18.5% | 301.12 | 23.8% |

Arithmetic Mean +/− SD

| | | | | | | |
|---|---|---|---|---|---|---|
| t_{1/2} (h) | 2.602 | 0.9800 | 3.696 | 0.9576 | 1.598 | N/C (n = 2) |
| CL (mL/h/kg) | 9.287 | 2.9419 | 10.835 | 2.1722 | 10.367 | N/C (n = 2) |
| Vdss (mL/kg) | 30.61 | 5.123 | 39.61 | 11.051 | 20.35 | N/C (n = 2) |
| MRT (h) | 3.568 | 1.2710 | 3.702 | 0.8641 | 1.961 | N/C (n = 2) |

Median (Min-Max)

| | | | | | | |
|---|---|---|---|---|---|---|
| tₘₐₓ (h) | 12.38 | (0.75-13.0) | 10.13 | (8.0-12.50) | 0.75 | (0.25-4.0) |

N/C = Not calculated when n < 3

Pharmacokinetic results show that the systemic exposure to ODSH was similar following the 3 dosing regimens. Mean clearance values of the 3 dosing regimens were similar, suggesting that the pharmacokinetics of ODSH is linear. Mean Cₘₐₓ values were comparable in both groups given the 8 mg/kg bolus (217 vs. 246 μg/mL). On the other hand, Cₘₐₓ values were greater following 16 mg/kg with the 32 mg/kg/12 hour infusion compared to the 8 mg/kg with the 32 mg/kg/12 hour infusion. The observed median tₘₐₓ values decreased from 12.4 to 10.1 hours when the infusion dose was increased 24 to 32 mg/kg/12 hour in the 8 mg/kg bolus regimens. Similarly, tₘₐₓ values deceased from 10.1 to 0.75 hours when the bolus dose was increased from 8 to 16 mg/kg in the 32 mg/kg/12 hour infusion regimens. This suggests that the 16 mg/kg loading dose of ODSH caused Cₘₐₓ to be reached at an earlier time point as compared to the other 2 treatments.

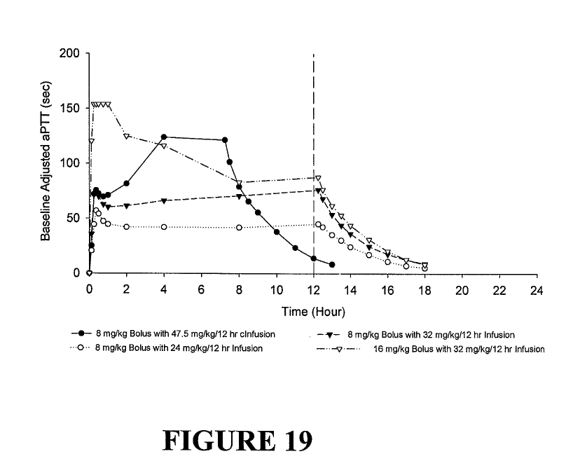

Mean values for aPTT for all groups are summarized in FIG. 19. ODSH bolus and infusion at the doses chosen induced sustained increases in aPTT over the 12 hour infusion period. Group 2 receiving a bolus of 8 mg/kg followed by an infusion of 24 mg/kg/12 hours (or 2 mg/kg/hr) experienced an immediate and sustained increase in aPTT of approximately 50 seconds above baseline (or on average an aPTT of about 75 to 80 seconds absolute), indicating that this dose (8 mg/kg bolus followed by 2 mg/kg/hr) would be useful to induce immediate therapeutic anticoagulation in subjects in need of this treatment. Subjects in group 1 (8 mg/kg bolus with 47.5 mg/kg/12 hour infusion) did not complete the 12-hour infusion because of a sustained elevation of aPTT of >120 seconds.

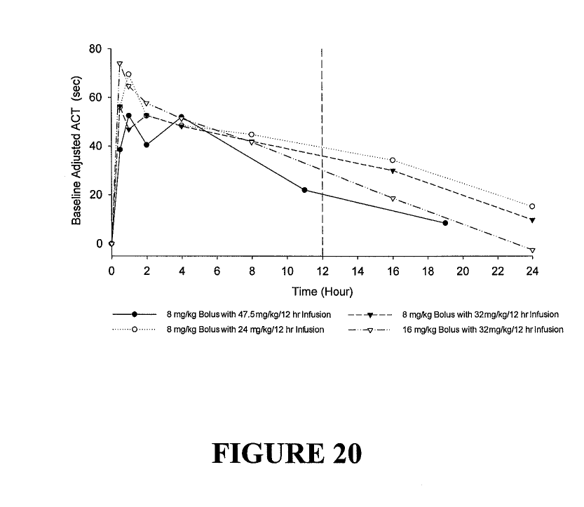

Mean values for ACT for all groups are summarized in FIG. 20. Increases in ACT were not as affected as aPTT by the amount of ODSH infused over 12 hours or by the intravenous loading dose. ACT increases providing adequate therapeutic anticoagulation necessary for the cardiac catheterization laboratory were observed in all dosing regimens.

Platelet counts for ODSH- and placebo-treated patients in all dose groups are shown below in Table 15, wherein platelet values are provided as thousands/μL blood (mean±SD). ODSH did not produce the >50% fall in platelet counts characteristic of heparin-induced thrombocytopenia (HIT), indicating that this heparin analog (ODSH) is safe from producing HIT during use at clinical doses in humans.

TABLE 15

| | Before Bolus | | 24 h After | |
|---|---|---|---|---|
| | Dose | | Bolus Dose | |
| Dose | ODSH | Placebo | ODSH | Placebo |
| 8 mg/kg bolus with | 285 ± 45 | | 256 ± 30 | |
| 47.5 mg/kg/12 hr | | | | |
| infusion | | | | |
| 8 mg/kg bolus with | 244 ± 40 | 306 ± 77 | 222 ± 39 | 267 ± 64 |
| 24 mg/kg/12 hr infusion | | | | |
| 8 mg/kg bolus with | 303 ± 63 | 242 ± 17 | 277 ± 62 | 205 ± 52 |
| 32 mg/kg/12 hr infusion | | | | |
| 16 mg/kg bolus with | 283 ± 50 | 227 ± 44 | 247 ± 43 | 213 ± 51 |
| 32 mg/kg/12 hr infusion | | | | |

The data provided in Table 15 demonstrate that ODSH is safe when administered in large boluses followed by infusion at doses which produce sustained anticoagulation. ODSH levels achieved in the dose group receiving a bolus of 8 mg/kg followed by 24 mg/kg/12 hr (2 mg/kg/hr) and therapeutically anticoagulated with an increase in aPTT of about 50 seconds above baseline were sustained at approximately 200 μg/ml plasma. Therefore, ODSH at this dose should be a safe drug for both producing therapeutic anticoagulation and inhibiting injurious intracellular Na⁺ and secondarily Ca⁺ overload from ischemia. Used in these bolus and infusion doses to produce therapeutic anticoagulation, ODSH also does not produce catastrophic thrombocytopenia characteristic of HIT.

### Example 6

### Safe Intravenous Bolus Administration and 72 Hour Infusion of 2-O, 3-O Desulfated Heparin to Normal Humans

A study was performed in eight (8) healthy adult male and female subjects to assess the effects of a bolus dose and 72 hour infusion of 2-O, 3-O desulfated heparin. The study was a Phase I study with a three day treatment period. Doses were adjusted to maintain an aPTT level of 40-45 seconds. Subjects were between the ages of 18 and 60, were not pregnant, and were normal in body weight. They all had normal coagulation function and hemoglobin values at baseline.

Subjects received an initial bolus of 8 mg/kg of ODSH over 15 minutes, followed by 72 hours continuous infusion beginning at 0.58 mg/kg/hr. For each subject ODSH as a 50 mg/ml formulation was diluted with normal saline and administered as a bolus infused over 15 minutes containing the calculated amount of ODSH the subject was to receive, followed by infusion of ODSH diluted in saline. The infusion dose was adjusted to maintain an aPTT of 40-45 seconds. Immediately before infusion and after the start of each infusion, blood was drawn at periodic times (over a total 72 hour period) to monitor the effect of infusion on the following laboratory studies: activated partial thromboplastin time (aPTT), prothrombin time (PT), activated clotting time (ACT), and ODSH plasma level. Serum chemistries and a complete blood count were checked immediately before infusion and again periodically for up to 240 hours later. Using values for aPTT and ODSH levels, pharmacokinetic parameters were calculated by noncompartmental methods using a commercial software program (PhAST 2.3-001). The following pharmacokinetic parameters were calculated as above: Cₘₐₓ; Kel; tₘₐₓ; AUC O-t; AUCinf, t_{1/2}; CL; and Vdss.

No serious adverse events occurred in this study and none of the subjects were discontinued from the study due to an adverse event. Specifically, bolus ODSH did not increase blood glucose, nor did it elevate blood pressure. Mild ecchymosis was reported in one subject and was assessed as unlikely to be related to ODSH. The infusion in two subjects was not able to be completed because of infusion pump mechanical failure. As commonly observed with therapeutic levels of unfractionated or low molecular weight heparins, transient elevations in serum alanine aminotransferase (ALT) and aspartate aminotransferase (AST) were observed in seven subjects, beginning on the third day of drug administration, peaking at day five or six, and returning to normal within two weeks. Such observations are reported by Dukes GE Jr., et al., Ann Int Med 100:646-650, 1984; and Carlson M K, et al., Pharmacotherapy 21:108-113, 2001.

There was no clear relationship to ODSH dose. In no case did ALT or AST rise to greater than seven times the upper limit of normal (ULN). Average peak ALT and AST was 3.1 times ULN. These transient elevations in tranaminases have been well-recognized to occur by regulatory agencies. The phenomenon is thought to be a class effect of all heparinoids and is not believed to be associated with adverse outcomes. Transaminase elevations from heparins were addressed in deliberations of a Canadian government scientific advisory panel on hepatotoxicity of health care products. Heparin was classified as an agent causing transaminasemia without significant liver damage. The Scientific Advisory Panel on Hepatotoxicity noted that heparin frequently causes an increase in transaminases after a few days of treatment but does not cause significant liver damage. The mechanism by which these agents increase transaminases is unknown, but the characteristics suggest a biochemical effect (Scientific Advisory Panel on Hepatotoxicity. Draft recommendations concerning “Recommendations from the Scientific Advisory Panel Sub-groups on Hepatotoxicity: Hepatotoxicity of Health Care Products”. Oct. 15, 2004. Available on-line at http://www.hs-sc.gc.ca/dhp-mps/prodpharma/activit/sci-consult/hepatotox/sap_gcs_hepatotox-2004-07-26_e.html).

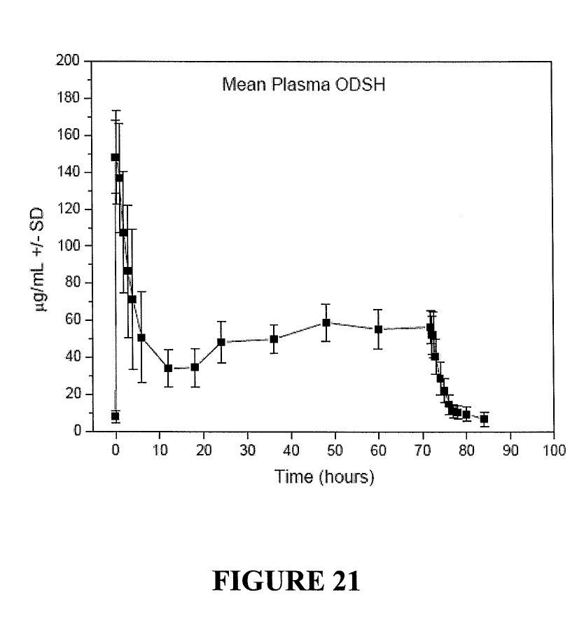

To achieve the goal of maintaining an aPTT of 40-45 seconds, the infusion rate was adjusted upward in all subjects so that subjects were infused with ODSH at 0.64 to 1.39 mg/kg/hr. The mean plasma ODSH concentrations for subjects is shown in FIG. 21. ODSH plasma concentrations near the end of infusion was approximately 50 μg/ml. Descriptive statistics of the pharmacokinetic parameters of ODSH in this study are summarized below in Table 16.

TABLE 16

| | |
|---|---|
| | ODSH Dose Levels |
| | 8 mg/kg Bolus with |
| | Infusion for 12 hr, |
| Pharmacokinetic | Final 0.64-1.39 mg/kg/hr |
| Parameters | (n = 6) |

| | | |
|---|---|---|
| Geometric Mean CV % | | |
| AUC | 4,053 | 9.9% |
| (μg h/mL) | | |
| Cₘₐₓ | 156 | 15.0% |
| (μg/mL) | | |
| Arithmetic Mean +/− SD | | |
| t_{1/2} (h) | 3.3 | ±1.0 |
| CL (mL/h/kg) | 10.2 | ±1.0 |
| Vdss (mL/kg) | 48.9 | ±16.0 |
| MRT (h) | 2.0 | ±2.8 |
| Median (Min-Max) | | |
| tₘₐₓ (h) | 0.5 | (0.25-0.5) |

Pharmacokinetic results showed that mean AUC was 4053 μg hr/mL, with a range of 3,528 to 4,694 μg hr/mL. Mean clearance value (CL) was 10.2 mL/hr/kg with a range from 8.8 to 11.8 mL/h/kg. The mean Cₘₐₓ was 156 μg/mL, with a range of 131 to 192 μg/mL. Mean Vdss was 48.9 mL/kg, with a range of 23.7 to 66.2 mL/kg. Median tₘₐₓ was 0.5 hours, with very little variation in the minimum to maximum range. The mean value of t_{1/2} was 3.3 hours, with a range of 1.9 to 4.4 hours. Mean MRT was 2.0 hours, with a range of −0.9 to 5.93 hours.

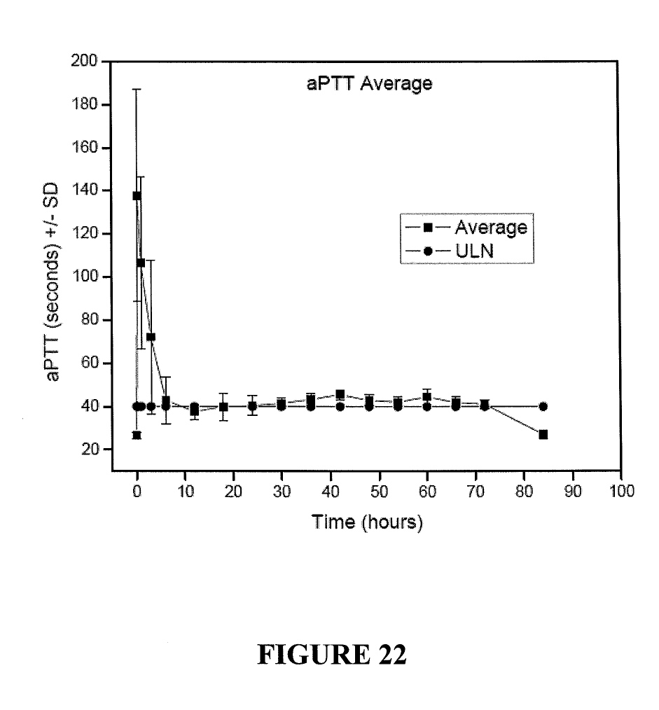

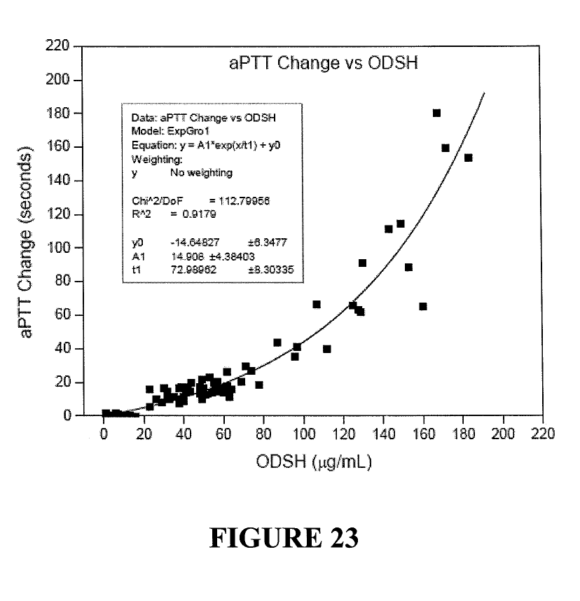

The mean aPTT in subjects over the 72 hours of study is shown in FIG. 22. ODSH produced a rapid increase in aPTT over the bolus infusion, but values fell to within the range of 40-45 seconds as the infusion was adjusted. The relationship between change in aPTT from baseline and ODSH levels for this study is shown in FIG. 23. This relationship illustrates that a therapeutic level of anticoagulation (approximately 50 seconds above baseline, or an absolute value of about 75 seconds) is achieved at ODSH blood concentrations of 100 μg/mL, which can inhibit injurious intracellular Na⁺ and secondarily Ca⁺⁺ overload from ischemia.

Platelet counts for the ODSH-treated subjects are shown below in Table 17. The table shows platelet counts after 8 gm/kg bolus followed by 72 hour infusion to aPTT of 40-45 seconds, with platelet values provided as thousands/μL blood (mean±SD). ODSH did not produce the >50% fall in platelet counts characteristic of heparin-induced thrombocytopenia (HIT), indicating that this heparin analog is safe from producing HIT during use at these clinical doses in humans.

TABLE 17

| Day Before | Infusion | Infusion | Day After |
|---|---|---|---|
| Infusion | Day 2 | Day 3 | Infusion |
| 261 ± 40 | 258 ± 39 | 272 ± 42 | 261 ± 37 |

These data demonstrate that ODSH is safe when administered at a bolus of 8 mg/kg followed by doses of 0.64 to 1.39 mg/kg/hr for 72 hours to maintain an aPTT of 40-45 seconds, producing sustained plasma ODSH levels of approximately 50 μg/mL. A therapeutic level of anticoagulation (approximately 50 seconds above baseline, or an absolute value of about 75 seconds) is achieved at ODSH blood concentrations of 100 μg/mL, which can maximally reduce or prevent injurious intracellular Na⁺ and secondarily Ca⁺⁺ overload from ischemia. Therefore, ODSH at this dose should be a safe drug for reducing or preventing injurious intracellular Na⁺ and secondarily Ca⁺⁺ overload from ischemia. Used in these doses, ODSH also does not produce catastrophic thrombocytopenia characteristic of HIT.

### Example 7

### Measurement of Na⁺ Channel Ionic Currents

This study was performed to probe another possible protection mechanism. Example 7 demonstrates that externally applied 2-O, 3-O desulfated heparin has direct effects on the cardiac myocyte sodium channel. Fused tsA201 cells (SV40 transformed HEK293 cells) expressing the cDNA for the human heart voltage-gated Na⁺ channel, Naᵥ1.5 (hH1a) were trypsinized and studied electrophysiologically as described previously (Sheets et al, 1996). For I_{Na} measurements the extracellular solution was (in mM): 15 Na⁺, 185 TMA⁺, 200 MES⁻, 10 HEPES, 3 CaOH₂, pH 7.2 with TMA-OH. The internal solution contained (in mM): 200 TMA⁺, 200 F⁻, 10 EGTA, and 10 HEPES (pH 7.2 by HF). For saxitoxin (STX, Calbiochem Corp., San Diego, Calif.) subtraction experiments, 1 μM STX was added to the extracellular solution, and 1 mM Ca²⁺ was added to all external solutions. The hypertonicity compensated for the lower conductivity of TMA⁺ and MES⁻ solutions. The Na⁺-free heparinic acid of ODSH was generated by passage over an ion exchange column, followed by lyophilization, and a concentration of 1 mg/ml was added, after which the pH was adjusted with TMA-OH.

I_{Na} current recordings were made with a large bore, double-barreled glass suction pipette for both voltage clamp and internal perfusion as previously described (Sheets et al, 1996). Currents were measured with a virtual ground amplifier (Burr-Brown OPA-101) using a 2.5 MΩ feedback resistor, and voltage protocols were imposed from a 16-bit DA converter (National Instruments, Austin, Tex.) over a 30/1 voltage divider. Data were filtered by the inherent response of the voltage-clamp circuit (corner frequency near 125 kHz) and recorded with a 16-bit AD converter at 200 kHz. A fraction of the current was fed back to compensate for series resistance. Cells were studied at room temperature. Leak resistance was calculated as the reciprocal of the linear conductance between −180 mV and −110 mV, and cell capacitance was measured from the integral of the current responses to voltage steps between −150 mV and −190 mV. Peak I_{Na} was taken as the mean of four data samples clustered around the maximal value of data digitally filtered at 5 kHz, and leak was corrected by the amount of the calculated time-independent linear leak. Data were capacity corrected using 4 to 8 scaled current responses recorded from voltage steps typically between −150 mV and −190 mV.

For peak I-V relationships, the holding membrane potential (Vₕₚ) was either −150 or −110 mV, step depolarizations were for 50 ms, and the pulse frequency was 0.5 sec. To account for any time-dependent shifts in I_{Na} kinetics, from a Vₕₚ of −150 mV the control values (closed circles) represent the means of the peak I_{Na} before ODSH heparinic acid and wash. From a Vₕₚ of −110 mV, to eliminate any leftward time-dependent shift in Na⁺ channel kinetics as a cause of a decrease in I_{Na} at −110 mV, peak I-V relationships were first recorded in ODSH heparinic acid before washing to control. Normalized peak I-V relationships were fit with a Boltzmann distribution:

I_{Na}=(Vₜ−Vᵣₑᵥ)Gₘₐₓ/(1+exp(Vₜ−V_{1/2}/s))

For steady-state voltage-dependent Na⁺ channel availability (SSI) curves the Vₕₚ was −150, the duration of the conditioning steps were 500 ms with a test step to 0 mV for 25 ms using an interpulse interval of 2.5 sec. To account for any leftward time-dependent shift in Na⁺ channel kinetics as a cause of a leftward shift in V_{1/2}, control values represent the means of peak I_{Na} before ODSH heparinic acid and after wash. Normalized steady-state voltage-dependent Na⁺ channel availability (SSI) curves were fit with a Boltzmann distribution:

I_{Na}=(Iₘₐₓ−Iᵣ)/(1+exp(Vc−V_{1/2}/s))+Iᵣ

For STX subtraction experiments the holding membrane potential was −110 mV, the test step duration was 100 ms, and the pulse frequency was 1 Hz. The 3 ms interval (from 96 ms to 99 ms in the test step) was averaged from the raw current recordings at each test potential for cells exposed to control solutions and to ODSH heparinic acid. The leak for each cell was measured by adding 1 μM STX to both the control and ODSH heparinic solutions, repeating the same voltage-clamp protocol, and meaning the data from the 3 ms interval between 96 to 99 ms of the 100 ms step. These leak values in STX were subtracted from those in control solutions. To account for any time dependent shift in kinetics, the late I_{Na} in control was taken as the mean of values measured before exposure to ODSH heparinic acid and after its wash except one of four cells only the wash measurements was used to compare to those in heparinic acid.

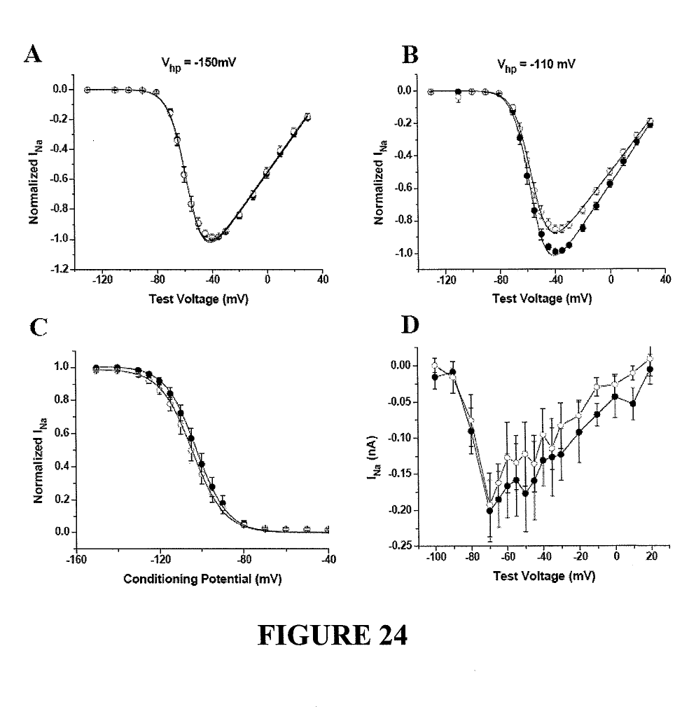

The above results in Example 1 provide strong, but indirect, evidence that ODSH is decreasing Na⁺ influx via I_{Na,L}. To provide more direct evidence for this effect, the influence of ODSH on Na⁺ channel current-voltage relationships was studied. The results of those experiments are shown in FIG. 24. In FIG. 24a, for n=6 cells, there were no significant differences between control and ODSH heparinic acid. Control values were; Gmax=1, V_{1/2}=−57±3 mV, slope (s)=−6.3±0.6. In ODSH heparinic acid values were; Gmax=1±0.04, V_{1/2}=−57±3 mV, slope (s)=−6.4±0.6. In FIG. 24b, For n=6 cells, Gₘₐₓ was significantly decreased from 1 to 0.87±0.05 in ODSH heparinic acid, the slope was not significantly changed (−6.1±0.7 in control vs. −6.2±0.8 in ODSH heparinic acid) while there was a small leftward shift in the V_{1/2} in control (−57±3 mV) compared to ODSH heparinic acid (−56±3 mV). In FIG. 24c, for n=6 cells, V_{1/2} was significantly shifted from −102±5 mV in control to −104±2 mV in ODSH heparinic acid. There were minimal but significant shift in the slope from 7.4±7 in control to 7.6±6 in ODSH heparinic acid but not in Iₘₐₓ from 1 to 0.99±0.01 in heparinic acid. In FIG. 24d, note the decrease in mean current in ODSH heparinic acid compared to control across the range of potentials. The values at −50, −40 and −30 mV were significantly different between control and ODSH heparinic acid. At a relatively high concentration of ODSH heparinic acid (1 mg/ml) the results indicate a rightward shift in the current-voltage relationship that results in a decrease in the inward Na⁺ current magnitude. The surprising demonstration is that ODSH significantly reduces reperfusion injury through ion channel effects early during reperfusion.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for controlling the intracellular calcium ion concentration in a subject, the method comprising administering an effective amount of an O-desulfated heparin to the subject prior to experiencing an ischemic event or while experiencing ischemia.

2. The method of claim 1, wherein the method comprises controlling calcium ion concentration in myocytes, neurons, renal cells, hepatocytes, or lung cells of the subject.

3. The method of claim 1, wherein the method further comprises reducing cellular influx of sodium ions.

4. The method of claim 1, wherein the O-desulfated heparin is desulfated at the 2-O position, the 3-O position, or both the 2-O and 3-O positions.

5. The method of claim 1, wherein the O-desulfated heparin is fully desulfated at the 2-O position and partially desulfated at the 3-O position.

6. The method of claim 1, wherein the O-desulfated heparin is partially desulfated at the 2-O position and fully desulfated at the 3-O position.

7. The method of claim 1, wherein the O-desulfated heparin comprises 2-O, 3-O desulfated heparin, wherein the heparin is at least about 10% desulfated at both the 2-O and 3-O positions.

8. The method of claim 1, wherein the O-desulfated heparin comprises 2-O, 3-O desulfated heparin, wherein the heparin is at least about 90% desulfated at both the 2-O and 3-O positions.

9. The method of claim 1, wherein the O-desulfated heparin is substantially sulfated at the 6-O position.

10. The method of claim 1, wherein the O-desulfated heparin comprises a molecular weight from 100 Da to 30,000 Da.

11. The method of claim 1, wherein the O-desulfated heparin comprises a molecular weight from 100 Da to 8,000 Da.

12. The method of claim 1, wherein the O-desulfated heparin comprises a molecular weight from 4,000 Da to 12,500 Da.

13. The method of claim 1, wherein the O-desulfated heparin comprises oxidized O-desulfated heparin, acetylated O-desulfated heparin, decarboxylated O-desulfated heparin, reduced O-desulfated heparin, or 6-O desulfated heparin.

14. The method of claim 1, further comprising administering one or more further bioactive agents for treating or preventing the effects of the ischemic event.

15. The method of claim 14, wherein the one or more further bioactive agents are administered sequentially

16. The method of claim 14, wherein the one or more further bioactive agents are administered concurrently.

17. The method of claim 14, wherein the further bioactive agent is selected from the group consisting of a glycoprotein IIb/IIIa inhibitor, aspirin, clopidogrel, a thrombolytic agent, a tissue plasminogen activator, a tissue reteplase, a tissue tenecteplase, a direct thrombin inhibitor, a Na⁺ channel inhibitor, a form of activated protein C, a fully anticoagulant unfractionated or low molecular weight heparin, and any combination thereof.

18. The method of claim 1, wherein the ischemic event comprises at least one of (1) a surgical interruption of blood flow, (2) a pathologic acute or subacute arterial occlusion from thrombosis of a blood vessel, (3) ligation of the blood vessel or vascular remodeling and proliferative overgrowth within the vessel wall, (4) exposure to low concentrations of oxygen in the blood stream, (5) a reduction in blood pressure, (6) cardiopulmonary arrest, or (7) a low concentration of red blood cells within the circulation of the subject.

19. The method of claim 1, wherein said controlling comprises reducing the intracellular calcium ion concentration in a subject experiencing ischemia.

20. The method of claim 1, wherein said controlling comprises maintaining the intracellular calcium ion concentration in a subject experiencing ischemia.

21. The method of claim 1, wherein said controlling comprises preventing an increase in the intracellular calcium ion concentration in a subject that is experiencing or is at risk of experiencing an ischemic event.

22. The method of claim 1, wherein said controlling comprises limiting an increase in the intracellular calcium ion concentration in a subject that is experiencing or is at risk of experiencing an ischemic event.

23. The method of claim 1, wherein the O-desulfated heparin is administered to the subject via intravenous administration, comprising administering O-desulfated heparin to the subject intravenously in an amount from about 1 mg/kg to about 20 mg/kg of subject body weight.

24. The method of claim 1, wherein the O-desulfated heparin is administered to the subject via an implantable medical device.

25. The method of claim 24, wherein the implantable medical device is coated with a composition comprising O-desulfated heparin.

26. The method of claim 24, wherein the implantable medical device is selected from the group consisting of a stent, catheter, balloon catheter, and shunt.

27. The method of claim 1, wherein the O-desulfated heparin is administered to the subject in one dose, at a controlled rate for a determined period of time, by repetitive intermittent administration, or a combination thereof.

28. A method for reducing the loss of function of a body part in a subject, the method comprising administering an effective amount of an O-desulfated heparin to the subject prior to experiencing an ischemic event or while experiencing ischemia.

29. The method of claim 28, wherein the body part is an organ selected from the group consisting of the heart, brain, lung, bowel, and kidneys.

30. The method of claim 28, wherein the body part is a body extremity.

31. The method of claim 28, wherein the loss of function is reduced by at least 10% compared to the loss of function in a subject not administered the O-desulfated heparin or a derivative thereof.

32. A method of treating one or more symptoms of ischemia, the method comprising administering to a subject an amount of an O-desulfated heparin effective to control the intracellular calcium ion concentration in the subject.

33. The method of claim 32, wherein the symptom is selected from the group consisting of (1) pain from vascular occlusion or disruption, (2) tissue destruction from necrosis or apoptosis, (3) an impairment in organ function, (4) an abnormal rhythm disturbance, and (5) a neurological impairment.

34. The method of claim 33, wherein the impairment in organ function is reduced during the ischemic event.
